SEQUENCE LISTING (6 sequences)

SEQ ID NO: 1 - DNA, 25 bp, Artificial Sequence
  Artificially synthesized
aggcctgctgaaaatgactgaatat

SEQ ID NO: 2 - DNA, 22 bp, Artificial Sequence
  Artificially synthesized
gctgtatcgtcaaggcactctt

SEQ ID NO: 3 - DNA, 16 bp, Artificial Sequence
  Artificially synthesized
  misc_feature (1)..(1): Amino acids Val Ile Cys (VIC) could be located prior to nucleic acid position 1
  misc_feature (16)..(16): Amino acids Met Gly Asn Phe Gln (MGBNFQ) could be located after nucleic acid position 16
ttggagctggtggcgt

SEQ ID NO: 4 - DNA, 15 bp, Artificial Sequence
  Artificially synthesized
  misc_feature (1)..(1): Amino acids Phe Ala Met (FAM)could be located prior to nucleic acid position 1
  misc_feature (15)..(15): Amino acids Met Gly Asn Phe Gln (MGBNFQ) could be located after nucleic acid position 15
tggagctgatggcgt

SEQ ID NO: 5 - DNA, 16 bp, Artificial Sequence
  Artificially synthesized
  misc_feature (1)..(1): Amino acids Phe Ala Met (FAM) could be located prior to nucleic acid position 1
  misc_feature (16)..(16): Amino acids Met Gly Asn Phe Gln (MGBNFQ) could be located after nucleic acid position 16
ttggagctcgtggcgt

SEQ ID NO: 6 - DNA, 13 bp, Artificial Sequence
  Artificially synthesized
  misc_feature (1)..(1): Amino acids Phe Ala Met (FAM) could be located prior to nucleic acid position 1
  misc_feature (13)..(13): Amino acids Met Gly Asn Phe Gln (MGBNFQ) could be located after nucleic acid position 13
gagctgttggcgt

(19) United States
(12) Patent Application Publication
(10) Pub. No.: US 2023/0142955 A1
(43) Pub. Date: May 11, 2023

(54) METHODS OF USING A MULTI-ANALYTE APPROACH FOR DIAGNOSIS AND STAGING A DISEASE

(71) Applicant: THE TRUSTEES OF THE UNIVERSITY OF PENNSYLVANIA, Philadelphia, PA (US)

(72) Inventors: David Aaron ISSADORE, Philadelphia, PA (US); Zijian YANG, Philadelphia, PA (US); Erica L. CARPENTER, Wyndmoor, PA (US); Ben Z. STANGER, Merion Station, PA (US)

(21) Appl. No.: 17/905,129

(22) PCT Filed: Feb. 26, 2021
(86) PCT No.: PCT/US2021/019900

§ 371 Date: Aug. 26, 2022

Related U.S. Application Data
(60) Provisional application No. 62/982,254, filed on Feb. 27, 2020.

(51) Int. Cl. C12Q 1/6886; C12N 15/10

(52) U.S. Cl. CPC C12Q 1/6886; C12N 15/1013; C12N 15/1089; C12Q 2600/156; C12Q 2600/158; C12Q 2600/178

(57) ABSTRACT

Disclosed herein are methods for evaluating a disease or a condition in a subject. More particularly, disclosed herein are methods for determining or diagnosing a disease, methods for classifying a stage of a disease, methods for treating a disease or methods for assessing the efficacy of a therapy for treating a disease based on the measurement and the computational analysis of various disease-specific biomarkers.

### CROSS REFERENCE TO RELATED APPLICATIONS

This application claims priority to and the benefit of U.S. Provisional Patent Application No. 62/982,254, filed Feb. 27, 2020, the disclosure of which is incorporated herein by reference in its entirety for any and all purposes.

### GOVERNMENT RIGHTS

This invention was made with government support under MH118170 awarded by the National Institutes of Health and W81XH-19-2-0002 awarded by the Army. The government has certain rights in the invention.

### SEQUENCE LISTING

This application contains a Sequence Listing which has been submitted electronically in ASCII format and is hereby incorporated by reference in its entirety. Said ASCII copy, created on Feb. 25, 2021, is named 103241.006706_SequenceListing.txt and is 3 Kilo bytes in size.

### TECHNICAL FIELD

The present disclosure relates to methods for evaluating a disease in a subject by measuring and performing computational analysis on a set of disease specific biomarkers.

### BACKGROUND

Pancreatic ductal adenocarcinoma (PDAC) is the third leading cause of cancer-related death in the United States, with an overall five-year survival of 9% (1). Diagnosis and staging currently rely on endoscopic ultrasound-guided biopsy, computerized tomography (CT), and magnetic resonance imaging (MRI) (2). Most patients are diagnosed at an advanced stage, and sufficiently sensitive and specific screening tests for early disease remain elusive. While curative-intent surgery remains an option for patients whose disease is confined to the pancreas, distinguishing these patients from those with metastases, who are unlikely to benefit from surgery, remains challenging due to the presence of occult metastases not detectable by standard of care imaging (3-5).

To address these challenges, several blood-based liquid biopsy biomarkers have been developed but show low sensitivity for detection of early stage disease (6-8). Carbohydrate antigen 19-9 (CA19-9), a longstanding PDAC-associated biomarker, is clinically utilized to monitor response to therapy but its role in screening or determining surgical resectability is unclear (9). More recently, several liquid biopsy biomarkers have shown potential for the diagnosis and staging of PDAC. Circulating cell-free DNA (ccfDNA) concentration has been shown to correlate with disease burden (10,11); KRAS mutations in ccfDNA have been detectable at various stages of disease although at lower rates in early stage disease (12,13); soluble protein biomarkers have demonstrated diagnostic value (14), and tumor-associated extracellular vesicles (EVs) have generated enthusiasm for their potential to improve diagnosis of the disease (7,14-16). Even with the current ongoing investigations, there remains a lack of sensitive assays for early detection of pancreatic cancer.

There is an urgent need to develop non-invasive methods for accurate and sensitive detection of a variety of diseases, disorders or conditions.

### SUMMARY

In meeting the described long-felt needs in the art, first provided herein are methods of determining whether a subject suffers from a disease or a condition. The methods comprise (a) measuring, in a processed sample from the subject, a set of circulating biomarkers comprising an extra-cellular vesicle (EV) miRNA, an EV mRNA, a circulating cell-free DNA, a circulating tumor DNA, and a protein biomarker specific for the disease or the condition; (b) applying a machine learning algorithm on the set of circulating biomarkers to generate an output indicative of a disease or a condition state of the subject; (c) determining whether the subject has the disease or the condition based upon the output so generated; and (d) treating the subject as needed.

Also provided herein are methods of classifying a stage of a disease or a condition in a subject in need thereof. The methods comprise (a) measuring, in a processed sample from the subject, a set of circulating biomarkers comprising an extra-cellular vesicle (EV) miRNA, an EV mRNA, a circulating cell-free DNA, a circulating tumor DNA, and a protein biomarker specific for the disease or the condition; (b) applying a machine learning algorithm on the set of circulating biomarkers to generate an output indicative of the stage of the disease or the condition of the subject; (c) determining the stage of the disease or the condition in the subject based upon the output so generated; and (d) recommending treatment or surgery for the subject.

Also provided herein are methods of assessing the efficacy of a therapy for treating a disease or a condition in a subject. The methods comprise (a) measuring, in a first processed sample taken from the subject before treatment, a set of circulating biomarkers comprising an extra-cellular vesicle (EV) miRNA, an EV mRNA, a circulating cell-free DNA, a circulating tumor DNA, and a protein biomarker specific for the disease or the condition; (b) measuring, in a second processed sample taken from the subject during or after treatment, the same set of circulating biomarkers from step (a); (c) applying a machine learning algorithm on the circulating biomarkers from step (a) and step (b) to generate a first output and a second output respectively indicative of a stage of the disease or the condition in the subject; and (d) determining a differential between the first output and second output, thereby assessing whether the efficacy of the therapy for treating the disease or the condition in the subject.

Further provided herein are methods of determining whether a subject suffers from a disease or a condition. The methods comprise (a) measuring, in a processed sample from the subject, a set of a plurality of circulating biomarkers selected by machine learning such that each biomarker is indicative of the disease or condition and such that the correlation between the circulating biomarkers is minimized; (b) generating an output, optionally by a machine learning algorithm, that is indicative of a disease or a condition state of the subject; (c) determining whether the subject has the disease or the condition based upon the output so generated; and (d) treating the subject as needed.

Further provided herein are methods of determining whether a subject suffers from a disease or a condition. The methods comprise (a) isolating a biological sample from the subject, using a magnetic separation filter device, wherein the magnetic separation filter device comprises a layer of magnetically soft material and a plurality of pores extending through the layer of magnetically soft material; (b) analyzing two or more biomarkers from the biological sample to generate an output; and (c) determining whether the subject has the disease or condition based upon the output so generated.

Also provided herein are methods of diagnosing a disease or condition in a subject. The methods comprise (a) isolating a biological sample from the subject, using a magnetic separation filter device, wherein the magnetic separation filter device comprises a layer of magnetically soft material and a plurality of pores extending through the layer of magnetically soft material; (b) analyzing two or more biomarkers from the biological sample to generate an output; and (c) diagnosing the disease or condition in the subject based upon the output so generated.

Also provided herein are methods of treating a disease or condition in a subject. The methods comprise (a) isolating a biological sample from the subject, using a magnetic separation filter device, wherein the magnetic separation filter device comprises a layer of magnetically soft material and a plurality of pores extending through the layer of magnetically soft material; (b) analyzing two or more biomarkers from the biological sample to generate an output; (c) diagnosing the disease or condition in the subject based upon the output so generated; and (d) administering a therapeutically effective amount of a drug suitable for treating the disease or condition to the subject.

In some embodiments, the disease or the condition is a cancer. In some embodiments, the cancer is a pancreatic cancer. In some embodiments, the pancreatic cancer is pancreatic ductal adenocarcinoma (PDAC). In other embodiments, the cancer is metastatic. In other embodiments, the cancer is non-metastatic.

In some embodiments, the biological sample comprises a plurality of extra-cellular vesicles (EV). In some embodiments, the plurality of extra-cellular vesicles are specific for the disease or condition. In some embodiments, the two or more biomarkers comprises EV miRNA or EV mRNA molecules. In some embodiments, the EV miRNA comprises hsa.miR.103b, hsa.miR.23a.3p, hsa.miR.409.3p, hsa.miR.224.5p, hsa.miR.1299, and any combinations thereof. In some embodiments, the EV mRNA comprises CD63, CK18, GAPDH, H3F3A, KRAS, ODC1, and any combinations thereof. In some embodiments, the analyzing of the two or more biomarkers comprises measuring an amount of the EV miRNA or EV mRNA molecules.

In some embodiments, the ccfDNA comprises an ALU repetitive element.

In some embodiments, the ctDNA comprises a mutated KRAS DNA with mutation KRASG12D, KRASG12V or KRASG12R.

In further embodiments, the two or more biomarkers further comprises a protein biomarker. In some embodiments, the protein biomarker is a cancer antigen protein. In other embodiments, the protein biomarker is cancer antigen 19-9 (CA19-9) protein. In some embodiments, the analyzing of the disclosed two or more biomarkers comprises measuring a concentration of the CA19-9 protein. In some embodiments, the two or more biomarkers further comprise a circulating cell-free DNA. In other embodiments, the analyzing two or more biomarkers comprises measuring a concentration of the circulating cell-free DNA. In some embodiments, the circulating tumor DNA comprises a mutated KRAS DNA. In further embodiments, the mutated KRAS DNA comprises a G12D, G12V or G12R mutation. In yet further embodiments, the circulating biomarkers comprise at least hsa.miR.1299, GAPDH mRNA, a mutated KRAS DNA and CA19-9 protein.

In other embodiments, the analyzing of the two or more biomarkers comprises sequencing, quantitative PCR, digital PCR, or immunoassay.

In some embodiments, the two or more biomarkers comprises an EV miRNA molecule selected from hsa.miR.103b, hsa.miR.23a.3p, hsa.miR.409.3p, hsa.miR.224.5p, and hsa.miR.1299; an EV mRNA molecule selected from CD63, CK18, GAPDH, H3F3A, KRAS, and ODC1; CA19-9 protein, a circulating cell-free DNA, a mutated KRAS DNA, or any combination thereof.

In other embodiments, the magnetic separation filter device is a track etched magnetic nanopore (TENPO) device. In some embodiments, the pores have an average diameter ranging from about 100 nm to 100 μm. In some embodiments, the pores the pores have an average diameter ranging from about 500 nm to about 25 μm. In some embodiments, the magnetic separation filter device comprises at least 1000 pores/mm². In some embodiments, the magnetically soft material comprises a nickel-iron alloy. In some embodiments, the magnetic separation filter device further comprises a layer comprising a material chosen from nickel and gold.

In some embodiments, the biological sample is taken from whole blood or plasma of the subject.

In further embodiments, the disclosed methods comprise applying a machine learning algorithm to the analyzing two or more biomarkers from the biological sample. In some embodiments, the machine learning algorithm comprises Least Absolute Shrinkage Selection Operator (LASSO). In some embodiments, the machine learning algorithm uses one or more classifier models selected from the group consisting of K-Nearest-Neighbors, SVM, linear discriminate analysis, logistic regression, Naive Bayes, and any combination thereof. In some embodiments, the machine learning algorithm distinguishes at least one of the two or more biomarkers from a control.

In other embodiments, the control comprises a reference value or circulating biomarkers from a healthy subject. In some embodiments, the isolating the biological sample comprise contacting the biological sample with an antibody. In some embodiments, the antibody comprise anti-human CD326, anti-human CD104, anti-human c-Met Monoclonal, anti-human CD44v6 antibody, anti-human TSPAN8, or any combination thereof

In yet other embodiments, the disclosed methods have an accuracy of more than 90% in identifying the disease or condition. In some embodiments, the accuracy is higher than a comparable method without the isolating the biological sample using the magnetic separation filter device.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a diagram illustrating how combining multiple circulating biomarkers allow diagnosing and staging PDAC. The present biomarker panel consists of the mRNA and miRNA cargo of tumor-derived EVs enriched from plasma, circulating CA19-9, cell-free circulating DNA concentration (as determined by qPCR to detect the ALU repeat element), and mutant KRAS allele fraction. This multiplex panel is combined algorithmically using machine learning. The system is trained using supervised learning on a cohort of 47 patients including 15 healthy individuals, 12 non-cancer disease controls, and 20 with various stages of PDAC. Finally, the developed classifiers are evaluated using an independent, blinded test set of 57 individuals to quantify performance.

FIGS. 2A-2D are series of graphs and heatmaps depicting the development of the biomarker panel using the training set. FIG. 2A: Heatmap shows values for the 14 circulating biomarkers from each patient in the training set, which included 15 healthy controls, 12 disease controls, and 20 PDAC patients. FIG. 2B: Fold changes of all biomarkers are plotted comparing PDAC vs. Non-Cancer patients. Error bars are standard deviation. ΔCq is calculated by Cq,PDAC—Cq,NC. FIG. 2C: Accuracy of each individual biomarker in PDAC diagnosis. Clinical threshold of 36 U/mL was used for CA19-9. Other biomarkers' thresholds were determined by Linear Discriminant Analysis. Error bars are standard error from bootstrapping 10 times from the training set. FIG. 2D: A colormap shows the Pearson correlation coefficient (R) between each circulating biomarker. The inset colormap shows the average Pearson correlation coefficient among EV-miRNAs (by averaging R from all possible EV-miRNA pairs), EV-mRNAs (by averaging R from all possible EV-mRNA pairs) with the CA19-9, ccfDNA concentration, and KRAS mutation detection in ctDNA.

FIGS. 3A-3H are series of charts and graphs demonstrating the applying the biomarker panel to distinguish PDAC from non-cancer. FIG. 3A A summary of the patient cohort used to train the dislcosed platform to classify PDAC vs. Non-PDAC. FIG. 3B We selected the panel using least absolute shrinkage and selection operator (LASSO). The best performing panel was selected based on its area under the curve (AUC) using 10-fold cross validation within the training set repeating 5 times. Error bars are standard error. FIG. 3C The resulting PDAC vs non-PDAC (PDAC-NC) panel consists of 5 biomarkers. FIG. 3D A learning curve generated by bootstrapping 10 times within the training set. Error bars are standard error. FIG. 3E A summary of the independent patient cohort used to validate the model in a blinded study. FIG. 3F The confusion matrix on the blinded test set showing that 28 of 30 non-PDAC samples (93.3%) and 24 of 27 PDAC samples (88.9%) were correctly identified. TPR: true positive rate; TNR: true negative rate; PPV: positive predictive value; NPV: negative predictive value; FIG. 3G Receiver operating characteristic (ROC) curve comparison between the 5-marker panel and the best individual biomarker CA19-9, plus two control experiments: 1. Random biomarkers, where the training set was used to generate a model without using feature selection. 2. Control, where the labels of the training data were randomized. Inset shows comparison of their AUCs. Error bars are standard error from bootstrapping 10 times. FIG. 3H Comparison of accuracy of the 5-marker panel and the best individual biomarkers. Control experiments are the same as described above. Error bars are standard error from bootstrapping 10 times.

FIGS. 4A-4G are series of charts and graphs depicting the retraining the model to distinguish metastatic from non-metastatic PDAC. FIG. 4A Patient cohort used to train the present platform to classify occult or imaging-confirmed metastatic patients from non-metastatic PDAC patients. Dotted line indicates one PDAC patient who was originally determined by imaging to be M0 but turned out to have TTM<4 months, hence was considered as occult metastases. FIG. 4B We selected the panel using least absolute shrinkage and selection operator (LASSO). The best performing panel was selected based on its AUC using 8-fold cross-validation within the training set and repeated 10 times. The inset shows the comparison of the accuracy between the presently disclosed panel (red) and the clinical diagnosis (grey). Error bars are standard error from bootstrapping 10 repeats. FIG. 4C The panel for metastatic PDAC detection consists of 4 biomarkers. FIG. 4D Learning curve of metastatic PDAC detection generated by bootstrapping N=10 times within the training set. Error bars represent standard error. FIG. 4E Proposed clinical workflow to combine liquid biopsy with imaging for a test set of 35 PDAC patients, including 8 patients who turned out to have TTM<4 months indicated by the dotted line. Baseline imaging was used to classify patients as either metastatic (Ml; N=12, top arm) or no detectable metastases (MOimaging; N=23, bottom arm). For the 23 MOimaging patients, the liquid biopsy panel was then performed, resulting in 2 patient classifications, those called by the model as M1 (occult metastases; top arm) or those called as M0 (MOLB; bottom arm). FIG. 4F Shown are the confusion matrices for the 23 PDAC MOimaging patients by imaging alone (bottom) and the present method combining liquid biopsy with machine learning (top). LB stands for liquid biopsy. The presently disclosed panel achieved accuracy=83%, with 75% sensitivity and 87% specificity. FIG. 4G Receiver operating characteristic (ROC) curve analysis on N=23 PDAC MOimaging patients in the blinded test set. Inset shows the accuracy comparison between imaging only (grey, accuracy=65%), control experiment (yellow, accuracy=46%), and liquid biopsy (red, accuracy=83%) panel. Error bars are standard error from bootstrapping 10 repeats.

FIG. 5 is series of tables listing the clinical characteristics of study population. * indicates 8 patients are included in the discovery as well as training sets. Designation of M0 versus M1 is based on baseline imaging

FIGS. 6A-6D are series of graphs and heatmaps depicting the miRNA sequencing to discover miRNA biomarkers to discern PDAC from non-cancer. FIG. 6A Raw miRNA sequencing data from 6 healthy controls, 6 non-cancer disease controls, 5 M0 PDAC patients, and 12 M1 PDAC patients. FIG. 6B 8 potential miRNA candidates were selected using least absolute shrinkage and selection operator (LASSO) for cancer versus non-cancer, achieving AUC=1 within discovery cohort. FIG. 6C We selected 5 out of 8 miRNA candidates based on their abundance as detected by qPCR (Cq<40) and show the average fold changes of these 5 miRNAs between patient groups. FIG. 6D We validated these 5 miRNAs by calculating the correlation coefficient between their qPCR data and the miRNA sequencing data. The overall R²=0.6.

FIG. 7 is a series of dot plots depicting individual biomarker profiles within the training set. 14 biomarkers' levels by patient group within the training set of 47 subjects. Pancreatic cancer patients (PDAC) relative to Non-Cancer patients (NC). Mann-Whitney test was used to evaluate statistical significance. * means P<0.05, ** means P<0.01, **** means P<0.0001.

FIG. 8 is a graph depicting the distribution of time to metastasis (TTM) for clinical M0 PDAC patients within the presently disclosed training and test sets. Cross indicates no metastasis observed in the last follow up, i.e., patient was censored at date of last follow up.

FIG. 9 is a series of pie charts depicting the sample cohort of this study, which included 133 subjects in total. Workflow shows patient cohorts involved in each classification. * indicates 8 patients included in both the discovery set and the training set.

FIG. 10 is a table listing the primers and probes used for KRAS mutation analysis (SEQ ID NOs: 1-6).

### DETAILED DESCRIPTION OF ILLUSTRATIVE EMBODIMENTS

The disclosed technology relates to, inter alia, methods for evaluating pancreatic cancer in a subject. More particularly, the disclosed technology relates to the field of determining pancreatic cancer, classifying a stage of pancreatic cancer or assessing the efficacy of a therapy for treating pancreatic cancer based on the measurement and the computational analysis of various biomarkers.

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e. , to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0, 1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia greata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

The terms “neurological diseases” or “neurological disorders”, as used herein, is used in the broadest sense and includes neurodegenerative diseases and disorders. As defined herein, a neurodegenerative disease or disorder may be characterized by the manifestation of gross physical dysfunction, not otherwise determinable as having emotional or psychiatric origins, typically resulting from progressive and irreversible loss of neurons. Such neurodegenerative diseases and disorders are defined in The Diagnostic and Statistical Manual of Mental Disorders-IV (DSM-IV) (American Psychiatric Association (1995)) and include, but are not limited to, Primary Lateral Sclerosis (PLS), Progressive Muscular Atrophy (PMA), Amyotrophic Lateral Sclerosis (ALS), Alzheimer's disease, Pick's disease, Huntington's disease, and Parkinson's disease. Of particular interest in the present invention are those diseases or disorders resulting from an alteration of normal SMN-associated processes including, but not limited to, SMA1 (Spinal Muscular Atrophy I, Werdnig-Hoffmann Disease, Infantile Muscular Atrophy), SMA2 (Spinal Muscular Atrophy II, Spinal Muscular Atrophy, Mild Child and Adolescent Form), SMA3 (Spinal Muscular Atrophy III, Juvenile Spinal Muscular Atrophy, Kugelberg-Welander Disease), and SMA4 (Spinal Muscular Atrophy IV).

The terms “psychiatric diseases” or “psychiatric disorders”, as used herein, may be characterized as one which is of emotional or psychiatric origin and is typically not associated with a loss of neurons. Exemplary psychiatric diseases and disorders include, but are not limited to, eating disorders, such as anorexia nervosa, bulimia nervosa, and atypical eating disorder; mood disorders, such as recurrent depressive disorder, bipolar affective disorder, persistent affective disorder, and secondary mood disorder; drug dependency such as alcoholism; neuroses, including anxiety, obsessional disorder, somatoform disorder, and dissociative disorder; grief; post-partum depression; psychosis such as hallucinations and delusions; dementia; paranoia; Tourette's syndrome; attention deficit disorder; psychosexual disorders, schizophrenia; and sleeping disorders.

The terms “dysregulated” and “dysregulation” as used herein describes a decreased (down-regulated) or increased (up-regulated) level of expression of a biomarker present and detected in a sample obtained from subject as compared to the level of expression of that biomarker present in a control sample, such as a control sample obtained from one or more normal, not-at-risk subjects, or from the same subject at a different time point. In some instances, the level of biomarker expression is compared with an average value obtained from more than one not-at-risk individuals. In other instances, the level of biomarker expression is compared with a biomarker level assessed in a sample obtained from one normal, not-at-risk subject.

“Differentially increased expression” or “up regulation” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween, than a control.

“Differentially decreased expression” or “down regulation” refers to expression levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1. 1 fold or less lower, and any and all whole or partial increments therebetween, than a control.

The term “expression” as used herein is defined as the transcription and/or translation of a particular nucleotide sequence.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, “microRNA” or “miRNA” describes miRNA molecules, generally about 15 to about 50 nucleotides in length, preferably 17- 23 nucleotides, which can play a role in regulating gene expression through, for example, a process termed RNA interference (RNAi). RNAi describes a phenomenon whereby the presence of an RNA sequence that is complementary or antisense to a sequence in a target gene messenger RNA (mRNA) results in inhibition of expression of the target gene. miRNAs are processed from hairpin precursors of about 70 or more nucleotides (pre-miRNA) which are derived from primary transcripts (pri-miRNA) through sequential cleavage by RNAse III enzymes.

By “nucleic acid” is meant any nucleic acid, whether composed of deoxyribonucleosides or ribonucleosides, and whether composed of phosphod jester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

Conventional notation is used herein to describe polynucleotide sequences: the left-hand end of a single-stranded polynucleotide sequence is the 5′- end; the left-hand direction of a double-stranded polynucleotide sequence is referred to as the 5′-direction.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

As used herein, “hybridization,” “hybridize (s)” or “capable of hybridizing” is understood to mean the forming of a double or triple stranded molecule or a molecule with partial double or triple stranded nature. Complementary sequences in the nucleic acids pair with each other to form a double helix. The resulting double-stranded nucleic acid is a “hybrid.” Hybridization may be between, for example two complementary or partially complementary sequences. The hybrid may have double-stranded regions and single stranded regions. The hybrid may be, for example, DNA:DNA, RNA:DNA or DNA:RNA. Hybrids may also be formed between modified nucleic acids (e.g., LNA compounds). One or both of the nucleic acids may be immobilized on a solid support. Hybridization techniques may be used to detect and isolate specific sequences, measure homology, or define other characteristics of one or both strands. The stability of a hybrid depends on a variety of factors including the length of complementarity, the presence of mismatches within the complementary region, the temperature and the concentration of salt in the reaction or nucleotide modifications in one of the two strands of the hybrid.

A “nucleic acid probe,” or a “probe”, as used herein, is a DNA probe or an RNA probe.

The term “Next-generation sequencing” (NGS), also known as high-throughput sequencing, is used herein to describe a number of different modern sequencing technologies that allow to sequence DNA and RNA much more quickly and cheaply than the previously used Sanger sequencing (Metzker, 2010, Nature Reviews Genetics 11.1: 31-46). It is based on micro- and nanotechnologies to reduce the size of sample, the reagent costs, and to enable massively parallel sequencing reactions. It can be highly multiplexed which allows simultaneous sequencing and analysis of millions of samples. NGS includes first, second, third as well as subsequent Next Generations Sequencing technologies.

“Sample” or “biological sample” as used herein means a biological material from a subject, including but is not limited to organ, tissue, exosome, blood, plasma, saliva, urine and other body fluid, A sample can be any source of material obtained from a subject. For instance the sample may comprise a cancerous pancreatic tissue sample, a benign pancreatic hyperplasia tissue, or a normal pancreatic tissue.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human. Non-human mammals include, for example, livestock and pets, such as ovine, bovine, porcine, canine, feline and murine mammals. Preferably, the subject is human. The term “subject” does not denote a particular age or sex. Preferably the subject is a human patient.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2,7, 3, 4, 5, 5.3, and 6. This applies regardless of the breadth of the range.

Illustrative Description

In one aspect, disclosed herein are methods of determining whether a subject suffers from a disease or a condition. The method comprises: (a) measuring, in a processed sample from the subject, a set of circulating biomarkers comprising an extra-cellular vesicle (EV) miRNA, an EV mRNA, a circulating cell-free DNA, a circulating tumor DNA, and a protein biomarker specific for the disease or the condition; (b) applying a machine learning algorithm on the set of circulating biomarkers to generate an output indicative of the disease or the condition state of the subject; (c) determining whether the subject has the disease or the condition based upon the output so generated; and (d) treating the subject as needed.

In one aspect, disclosed herein are methods of determining whether a subject suffers from a disease or a condition. The methods comprise (a) isolating a biological sample from the subject, using a magnetic separation filter device, wherein the magnetic separation filter device comprises a layer of magnetically soft material and a plurality of pores extending through the layer of magnetically soft material; (b) analyzing two or more biomarkers from the biological sample to generate an output; and (c) determining whether the subject has the disease or condition based upon the output so generated.

In some embodiments, the determining whether a subject suffers from a disease or a condition or the identifying of a disease or a condition has an accuracy of more than 90% or at least 90%. In some embodiments, the determining whether a subject suffers from a disease or a condition has an accuracy of at least 60%, at least 65%, at least 70%, at least 75%, at least 80%, at least 85%, at least 90%, at least 95%, at least 98% or more. In some embodiments, the accuracy is higher than a comparable method without isolating a biological sample using a magnetic separation filter device. In some embodiments, the determining whether a subject has pancreatic cancer has an accuracy of at least 60%, at least 65%, at least 70%, at least 75%, at least 80%, at least 85%, at least 90%, at least 95%, at least 98% or more.

In some embodiments, the determining whether a subject suffers from a disease or a condition comprises a sensitivity of about 75% and specificity of about 87%. In some embodiment, the sensitivity is at least 60%, at least 65%, at least 70%, at least 75%, at least 80%, at least 85%, at least 90%, at least 95%, at least 98% or more. In some embodiment, the specificity is at least 60%, at least 65%, at least 70%, at least 75%, at least 80%, at least 85%, at least 90%, at least 95%, at least 98% or more.

In one aspect, disclosed herein are methods of classifying a stage of a disease or a condition in a subject in need thereof. The method comprises: (a) measuring, in a processed sample from the subject, a set of circulating biomarkers comprising an extra-cellular vesicle (EV) miRNA, an EV mRNA, a circulating cell-free DNA, a circulating tumor DNA, and a protein biomarker specific for the disease or the condition; (b) applying a machine learning algorithm on the set of circulating biomarkers to generate an output indicative of the stage of the disease or the condition in the subject; (c) determining the stage of the disease or the condition in the subject based upon the output so generated; and (d) recommending treatment or surgery for the subject.

In one aspect, disclosed herein are methods of assessing the efficacy of a therapy for treating pancreatic cancer in a subject. The method comprises: (a) measuring, in a first processed sample taken from the subject before treatment, a set of circulating biomarkers comprising an extra-cellular vesicle (EV) miRNA, an EV mRNA, a circulating cell-free DNA, a circulating tumor DNA, and a protein biomarker specific for the disease or the condition; (b) measuring, in a second processed sample taken from the subject during or after treatment, the same set of circulating biomarkers from step (a); (c) applying a machine learning algorithm on the circulating biomarkers from step (a) and step (b) to generate a first output and a second output respectively indicative of a stage of the disease or the condition in the subject; and (d) determining a differential between the first output and second output, thereby assessing whether the efficacy of the therapy for treating the disease or the condition in the subject.

In one aspect, disclosed herein are methods of determining whether a subject suffers from a disease or a condition. The method comprise: (a) measuring, in a processed sample from the subject, a set of a plurality of circulating biomarkers selected by machine learning such that each biomarker is indicative of the disease or condition and such that the correlation between the circulating biomarkers is minimized; (b) generating an output, optionally by a machine learning algorithm, that is indicative of a disease or a condition state of the subject; (c) determining whether the subject has the disease or the condition based upon the output so generated; and (c) treating the subject as needed.

In one aspect, disclosed herein are methods of diagnosing a disease or condition in a subject. The methods comprise (a) isolating a biological sample from the subject, using a magnetic separation filter device, wherein the magnetic separation filter device comprises a layer of magnetically soft material and a plurality of pores extending through the layer of magnetically soft material; (b) analyzing two or more biomarkers from the biological sample to generate an output; and (c) diagnosing the disease or condition in the subject based upon the output so generated.

In one aspect, disclosed herein are methods of treating a disease or condition in a subject. The methods comprise (a) isolating a biological sample from the subject, using a magnetic separation filter device, wherein the magnetic separation filter device comprises a layer of magnetically soft material and a plurality of pores extending through the layer of magnetically soft material; (b) analyzing two or more biomarkers from the biological sample to generate an output; (c) diagnosing the disease or condition in the subject based upon the output so generated; and (d) administering a therapeutically effective amount of a drug suitable for treating the disease or condition to the subject.

In some embodiments, the correlation between biomarkers is less than 0.75, less than 0.65, less than 0.6, less than 0.55, less than 0.5, less than 0.45, less than 0.4, less than 0.35, less than 0.3, less than 0.25, less than 0.2, less than 0.15, less than 0.1, or less than 0.05. In some embodiments, the correlation between biomarkers is less 0.6.

In various embodiments of the disclosed methods, the disease or the condition is a cancer. In some embodiments, the cancer is a pancreatic cancer. In other embodiments, the cancer is at a metastatic stage. In yet other embodiments, an absence of metastasis is an indication that a treatment or a surgery is beneficial for the subject.

In some embodiments, the biological sample comprises a plurality of extra-cellular vesicles (EV). In some embodiments, the plurality of extra-cellular vesicles are specific for the disease or condition. In some embodiments, the two or more biomarkers comprises EV miRNA or EV mRNA molecules. In some embodiments, the EV miRNA comprises hsa.miR.103b, hsa.miR.23a.3p, hsa.miR.409.3p, hsa.miR.224.5p, hsa.miR.1299, and any combinations thereof. In some embodiments, the EV mRNA comprises CD63, CK18, GAPDH, H3F3A, KRAS, ODC1, and any combinations thereof. In some embodiments, the analyzing of the two or more biomarkers comprises measuring an amount of the EV miRNA or EV mRNA molecules.

In yet other embodiments, the ccfDNA comprises an ALU repetitive element.

In further embodiments, the ctDNA comprises a mutated KRAS DNA with mutation KRASG12D, KRASG12V or KRASG12R.

In some embodiments, the protein biomarker is a cancer antigen protein. In some embodiments, the protein biomarker is cancer antigen 19-9 (CA19-9) protein.

In some embodiments, the circulating biomarkers comprise at least hsa.miR.1299, GAPDH mRNA, a mutated KRAS DNA and CA19-9 protein. In other embodiments, the disclosed two or more biomarkers comprise an EV miRNA molecule selected from hsa.miR.103b, hsa.miR.23a.3p, hsa.miR.409.3p, hsa.miR.224.5p, and hsa.miR.1299; an EV mRNA molecule selected from CD63, CK18, GAPDH, H3F3A, KRAS, and ODC1; CA19-9 protein, a circulating cell-free DNA, a mutated KRAS DNA, or any combination thereof

An amount of biomarker in the biological sample can be measured or quantified by any known RNA, DNA or protein detection methods. In some embodiments, the analysis of the disclosed two or more biomarkers comprises measuring a concentration of the circulating cell-free DNA. In some embodiments, the circulating tumor DNA comprises a mutated KRAS DNA. In further embodiments, the mutated KRAS DNA comprises a G12D, G12V or G12R mutation.

In some embodiments, the sample is taken from whole blood or plasma. The sample may be of any biological tissue or fluid. In some embodiments, the sample can be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. In some embodiments, isolating the biological sample comprise contacting the biological sample with an antibody. In some embodiments, the antibody comprise anti-human CD326, anti-human CD104, anti-human c-Met Monoclonal, anti-human CD44v6 antibody, anti-human TSPAN8, or any combination thereof

In some embodiments, the processed sample comprises extracted, amplified and/or labeled DNA, RNA or protein.

Detection of protein-based biomarkers includes, but is not limited to, sequencing, quantitative PCR, digital PCR, two-dimensional electrophoresis, mass spectrometry and immunoassay. An antigen or antibody can be assessed for immunospecific binding by any method known in the art. The immunoassays that can be used include but are not limited to competitive and non-competitive assay systems using techniques such as western blots, radioimmunoassays, ELISA (enzyme linked immunosorbent assay), sandwich immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few.

In some embodiments, nucleic acids (e.g. miRNA, mRNA or DNA) in a biological sample can be detected or read by a sequencing method (including Sanger sequencing, next-generation sequencing or deep sequencing, direct multiplexing, and any art-recognized sequencing method) and a read count of each sequence can be generated to determine its amount present in the biological sample. In other embodiments, nucleic acids of interest can be assessed by, but not limited to, PCR, digital PCR, quantitative RT-PCR applications, microarray platforms or bead-based flow cytometric expression profiling methods. Any other art-recognized methods detecting or measuring the level of a nucleic acid sequence can also be used herein. In some embodiments, the one or more of the circulating biomarkers disclosed herein are measured by one or more of sequencing, quantitative PCR, digital PCR, or immunoassay.

In some embodiments, the biological sample from the subject is isolated by using a magnetic separation filter device. In some embodiments, the magnetic separation filter device comprises a layer of magnetically soft material and a plurality of pores extending through the layer of magnetically soft material. In other embodiments, the magnetic separation filter device is a track etched magnetic nanopore (TENPO) device. In some embodiments, the pores have an average diameter of about: 50nm, 100 nm, 150 nm, 200 nm, 250 nm, 300 nm, 350 nm, 400 nm, 450 nm, 500 nm, 550 nm, 600 nm, 650nm, 700 nm, 750 nm, 800 nm, 850 nm, 900 nm, 950 nm, 1μm, 25 um, 50 um, 75 um, 100 um, 125 um, 150 μm, 175 μm or 200 um. In some embodiments, the pores have an average diameter ranging from about 100 nm to 100 um. In some embodiments, the pores the pores have an average diameter ranging from about 500 nm to about 25 μm. In some embodiments, the magnetic separation filter device comprises at least: 800 pores/mm², 900 pores/mm², 1000 pores/mm², 1100 pores/mm², 1200 pores/mm², 1300 pores/mm², 1400 pores/mm² or 1500 pores/mm². In embodiments, the magnetic separation filter device comprises at least 1000 pores/mm². In some embodiments, the magnetically soft material comprises a nickel-iron alloy. In some embodiments, the magnetic separation filter device further comprises a layer comprising a material chosen from nickel and gold. In other embodiments, the EV miRNA and EV mRNA are extracted by a TENPO device.

In some embodiments, the methods provided herein can be useful for a wide variety of diseases, disorders, and conditions including, but not limited to, cancer, autoimmune diseases, neurological disorders, psychiatric disorders and acute or chronic infections such as viral, bacterial, parasitic and fungal infections.

In some embodiments, the methods provided herein can useful for a variety of cancers. These include solid or metastatic tumors. In some embodiments, the cancer is metastatic. In other embodiments, the cancer is non- metastatic. Metastasis is a form of cancer wherein the transformed or malignant cells are traveling and spreading the cancer from one site to another. Such cancers include cancers of the skin, breast, brain, cervix, testes, etc. More particularly, cancers can include, but are not limited to the following organs or systems: cardiac, lung, gastrointestinal, genitourinary tract, liver, bone, nervous system, gynecological, hematologic, skin, and adrenal glands. More particularly, the methods herein can be used for treating gliomas (Schwannoma, glioblastoma, astrocytoma), neuroblastoma, pheochromocytoma, paraganlioma, meningioma, adrenalcortical carcinoma, kidney cancer, vascular cancer of various types, osteoblastic osteocarcinoma, prostate cancer, ovarian cancer, uterine leiomyomas, salivary gland cancer, choroid plexus carcinoma, mammary cancer, pancreatic cancer, pancreatic ductal adenocarcinoma (PDAC), colon cancer, and megakaryoblastic leukemia. Skin cancer includes malignant melanoma, basal cell carcinoma, squamous cell carcinoma, Karposi's sarcoma, moles dysplastic nevi, lipoma, angioma, dermatofibroma, keloids, and psoriasis. In other embodiments, the cancer treated by the presently disclosed methods comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an oesophagus cancer or a soft tissue cancer. In one embodiment, the cancer is a pancreatic cancer. In another embodiment, the pancreatic cancer is pancreatic ductal adenocarcinoma (PDAC).

In some embodiments, the presently disclosed methods include computational analysis based on a machine learning data analysis. The analysis can comprise a selection step, a training step (e.g. by Least Absolute Shrinkage and Selection Operator (LASSO)), and a validation step using a blinded test set. In some embodiments, various machine learning algorithms can be used. These include but are not limited to K-Nearest-Neighbors, SVM, linear discriminate analysis, logistic regression, and Naive Bayes). In some embodiments, the output results are averaged. In other embodiments, a bootstrapping method can be applied.

In some embodiments, the machine learning algorithm distinguishes the circulating biomarkers from a control. In some embodiments, the machine learning algorithm distinguishes at least one of the two or more biomarkers from a control.

In some embodiments, the control comprises a reference value or circulating biomarkers from a healthy subject. In other embodiments, the control comprises circulating biomarkers from a subject without a cancer or with a non-metastatic cancer.

Reference Value or Control

The methods provided herein include comparing and distinguishing the circulating biomarkers from a control comprising a reference value, circulating biomarkers from a healthy subject or circulating biomarkers from a subject without cancer or with non-metastatic cancer. Preferably, the healthy subject is a subject of similar age, gender and race and has never been diagnosed with any type of disease, disorder or condition.

In another embodiment, the reference value of the biomarkers of interest is a value for expression of these biomarkers that is accepted in the art. This reference value can be baseline value calculated for a group of subjects based on the average or mean values of biomarkers by applying standard statistically methods.

In certain aspects of the present invention, the level of biomarkers is determined in a sample from a subject. The sample can include diseased cells, degenerating cells, tumor cells, any fluid from the surrounding of diseased, degenerating or tumor cells (e.g. blood, or tumor tissue) or any fluid that is in physiological contact or proximity with the diseased or tumor cells, or any other body fluid in addition to those recited herein should also be considered to be included herein.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

Materials and Methods

Patients and Sample Collection and Processing

Whole blood was collected at baseline (therapy-naïve) from 133 total patients at the Hospital of the University of Pennsylvania after obtaining written informed consent. Among the 67 patients with PDAC, 36 were clinically staged as having local disease only (M0), including 28 resectable patients and 8 patients with locally advanced disease. The determination of locally advanced disease was made either at the time of baseline imaging or intra-operatively due to vascular involvement. The remaining 31 patients had imaging-confirmed metastatic disease (M1; FIG. 5 and FIG. 9).

For the staging analysis, retrospective chart review was conducted to determine whether 32 patients originally staged by imaging as metastasis-free (M0) might have harbored metastatic disease below the level of detection for standard of care imaging. Among 32 M0 patients, 9 were categorized as having had occult metastases, including 4 with metastases detected intra-operatively and 5 with very early recurrence, here defined as within 4 months of baseline blood draw. Time to metastasis (TTM) was defined with respect to the date of baseline blood draw, censoring patients based on the date of last follow-up. Imaging data and clinical staging were obtained by chart abstraction. The 66 subjects serving as non-cancer controls included 26 patients with non-cancer pancreatic diseases such as intraductal papillary mucinous neoplasm (IPMN) and pancreatitis, as well as 40 healthy individuals enrolled at the time of routine screening procedures such as colonoscopy or endoscopy. Patients with an active malignancy at the time of blood draw were excluded from the control cohorts. All non-cancer control patients were followed for a minimum of 4 months to verify that no patient received a PDAC diagnosis subsequent to blood draw. Venous blood was collected in K2EDTA (Becton Dickinson) or Streck cfDNA BCT (Streck) tubes and processed to plasma as previously described (17). K2EDTA and Streck cfDNA whole blood was processed within 3 or 24 hours after blood draw, respectively. Plasma was aliquoted and stored at −80° C. for future use. All subjects had sufficient total plasma from a single blood draw such that all assays described below could be performed. Study was designed and conducted in accordance with the Reporting recommendations for tumor MARKer prognostic studies (REMARK) guidelines (20).

Tumor Derived EV miRNA and mRNA Isolation by Track Etched Magnetic Nanopore (TENPO) Device

EVs from each patient's K2EDTA-collected plasma (1.5 mL) were magnetically labeled using biotinylated antibodies and anti-biotin ultrapure 50nm diameter nanoparticles (Miltenyi Biotec). Antibodies used in this study included anti-human CD326 (EpCAM) (BioLegend), anti-human CD104 (ThermoFisher Scientific), anti-human c-Met Monoclonal (ThermoFisher Scientific), anti-human CD44v6 antibody (ThermoFisher Scientific), and anti-human TSPAN8 (Miltenyi Biotec). These surface markers have been previously shown to enrich pancreatic tumor-associated EVs from plasma (17,21). These five biotinylated antibodies (1.25 μL each) were pipetted into the human plasma samples and incubated for 20 minutes at room temperature on a shaking mixer. Subsequently, anti-biotin magnetic nanoparticles (20 μL, Miltenyi Biotec) were added to the samples and incubated for another 20 minutes at room temperature on the shaking mixer. Next, the plasma samples were loaded into the reservoir of the TENPO device which was connected to a programmable syringe pump (Braintree Scientific) to provide the negative pressure driving the sample through the device.

Details on the design and fabrication of TENPO have been previously reported (17). Briefly, a permanent magnet (NdFeB disc magnet, d=1.5 inches,h=0.75 inches, K&J Magnetics) was placed beneath the TENPO device to magnetize TENPO's paramagnetic Ni₈₀Fe₂₀ film and the superparamagnetic nanoparticles used to label the EVs. While samples were pulled through the device, EVs that were labeled with a sufficient number of magnetic nanoparticles were captured at the edges of the chip's nanopores, while background EVs flowed through and were discarded. The positively selected EVs were subsequently lysed on the chip by directly loading QIAzol lysis reagent (700 mL, Qiagen) on chip, incubated for 3 minutes, and collected the lysate. The RNA was then extracted from this lysate off-chip (ExoRNeasy serum/plasma kit, Qiagen). The EV miRNAs and mRNAs were eluted and stored at −80° C. or immediately processed for further analysis.

EV miRNA Sequencing and Candidate Discovery

A discovery cohort of 29 samples (FIG. 5, FIG. 9) was analyzed by next-generation sequencing to identify miRNAs in the enriched tumor associated EVs that might be differentially expressed among patient cohorts. QlAseq miRNA library kit (Qiagen) was used to make a library from isolated EV miRNA. A BioAnalyzer was used to quantify RNA prior to sequencing. The library was sequenced using a HiSeq 2500 kit (Illumina, Next-Generation Sequencing Core, University of Pennsylvania). A modified version of the UPenn SCAP-T RNA-Seq expression pipeline (Fisher, S A., “Safisher/Ngs.” GitHub, 2017) was used for expression quantification by aligning to the hg38 genomes. The minimum fragment length allowed past the TRIM module was adjusted to 16 bases for miRNA analysis. The number of allowed mismatches was capped at one and unannotated splices were prohibited. Expression counts were normalized by DESeq2 (22) and quantified using VERSE (23), using Gencode 25 and UCSD mm10 gene annotations, combined with MirBase v21 annotations for 3p and 5p microRNA.

Selection of EV RNA Panel

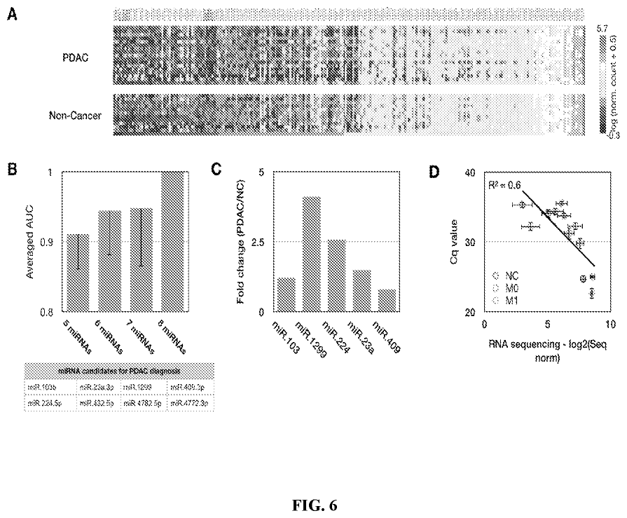

To identify potential EV miRNA candidates for PDAC diagnosis, the feature selection algorithm Least Absolute Shrinkage and Selection Operator (LASSO) was applied on EV miRNA sequencing results to find the most informative miRNAs (FIG. 6A). The resulting eight miRNA candidates were: hsa.miR.103b, hsa.miR.23a.3p, hsa.miR.432.5p, hsa.miR.409.3p, hsa.miR.224.5p, hsa.miR.1299, hsa.miR.4782.5p, and hsa.miR.4772.3p (FIG. 6B). Next, the miRNA candidates were validated by qPCR, and 3 miRNAs (hsa.miR.4772.3p, hsa.miR.4782.5p, and hsa.miR.432.5p) were identified with Cq which were considered to not be adequately abundant and were therefore excluded from further analysis (FIG. 6C). The remaining five miRNAs were measured by qPCR within the training set (N=47) and were compared with the EV miRNA sequencing data (FIG. 6D) within each patient subset (non-cancer and PDAC). The qPCR and sequencing data corresponded well with one another (R²=0.6, FIG. 6D). Six EV mRNAs (CD63, CK18, GAPDH, H3F3A, KRAS, ODC1) were also included. These had previously been used to distinguish stage IV PDAC patients from healthy controls (17) to form a panel of 11 potential EV RNA biomarkers. These 11 EV RNA biomarkers combined with CA19-9, ccfDNA concentration (qPCR for ALU), and ctDNA (KRAS mutation allele fraction) formed the final 14-biomarker-candidates for later classification.

EV miRNA and mRNA qPCR

The miScript SYBR Green PCR kit (Qiagen) and miScript primers (Qiagen) were used to quantify EV miRNAs. A master mix containing miScript SYBR Green, miScript primer, universal primer, and RNase-free water was prepared at a 5:1:1:2 ratio. 9 μl of the master mix was added to each well of a 384-well plate, followed by 1 μl of cDNA. 40 cycles were run with a default setting using CFX384 Touch Real-Time PCR machine (Bio-Rad). The SsoAdvanced Universal SYBR Green Supermix (Bio-Rad) and primers (Integrated DNA Technologies) were used for EV mRNA quantification. The SYBR Green supermix, primers, and RNase-free water were combined at a 5:0.5:3.5 ratio for the master mix. 9 μl of the master mix was added to each well, followed by 1 μl of cDNA. 40 cycles were run with a default setting using CFX384 Touch Real-Time PCR machine (Bio-Rad). Duplicates were performed for each sample. The melting curves for the amplified DNA were manually validated before subsequent analysis.

ccfDNA Extraction and Concentration

ccfDNA was isolated from K2EDTA- or Streck-collected plasma. If necessary to ensure a consistent input volume across all samples, the volume was adjusted with Phosphate Buffered Saline and the measured ccfDNA concentration was corrected for original input. Extraction was performed using the QlAamp Circulating Nucleic Acid Kit (Qiagen #55114) with two modifications to the manufacturers protocol. First, incubation of the buffer-lysate solution was increased to 1 hour at 60° C. Second, the final elution was carried out twice with 30 μL of Buffer AVE for a total of 60 μL. The extracted ccfDNA from 1 mL of plasma was used for downstream assays with extracted ccfDNA stored at 4° C. for short-term use or at −20° C. for long-term storage. The concentration of extracted ccfDNA was quantified by qPCR for a 115 bp amplicon of the ALU repetitive element. Briefly, qPCR was carried out on 1 μL of extracted ccfDNA, in quadruplicate, using Power SYBR Green PCR Master Mix (Applied Biosystems #4367659) according to the manufacturer's instructions on a ViiA 7 Real-Time PCR System (Applied Biosystems). Results were normalized to a standard curve of reference DNA (Promega #PAG3041) using QuantStudio Real-Time PCR Software (Applied Biosystems).

Pre-amplification ddPCR for Detection of Circulating KRAS G12DN/R Mutations

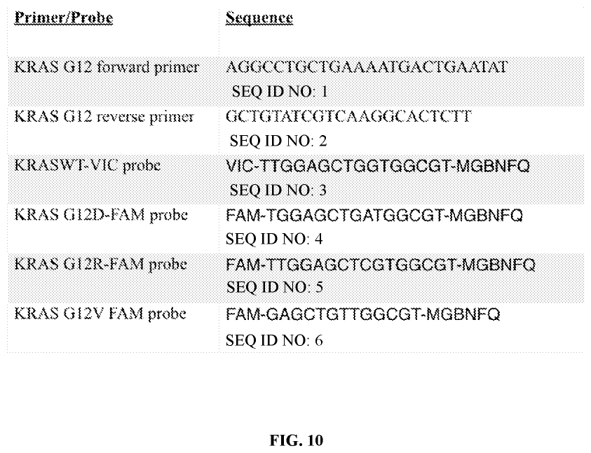

Pre-amplification PCR of the KRAS G12 locus was performed using 15 μL of ccfDNA eluate in a 50 μL reaction. Pre-amplified material was diluted 1:4 with TE buffer and stored for short-term use at 4° C. and at −20° C. for long-term storage. Multiplex ddPCR to detect KRAS G12DN/R/WT or duplex ddPCR (KRAS G12D/WT, G12V/WT, or G12R/WT) was prepared as a 30 μL reaction mix containing 2× TaqMan Genotyping Master Mix, lx droplet stabilizer, and 200 nM primers (FIG. 10; SEQ ID NOs: 1-6), probes at 50 nM (multiplex G12R only) or 100 nM (multiplex G12D and WT, both probes in duplex assays), and 100 μL of diluted pre-amplification reaction. Multiplex ddPCR for KRAS G12DN/R/WT was initially used to identify positive samples; these findings were verified and quantified by testing with identified variant's specific duplex assay. 25 μL of each reaction mix was loaded onto the RainDrop Source instrument (RainDance Technologies, Inc.) for droplet production. Mutant allele fraction was calculated as the mutant allele copy number divided by the total (wild-type+mutant) copy number. Samples that failed to meet mutant copy number thresholds or with a mutant allele fraction <0.01% were considered undetectable and assigned a value of 0.001%. Of the samples with a detectable KRAS mutation, the allele fraction was analyzed as a continuous variable, with values ranging from 0.01%-39.08% (median 0.405%).

CA19-9 Measurement

The Hospital of the University of Pennsylvania Clinical Immunology Laboratory was provided a 200 ul aliquot of K2EDTA plasma that had been banked at −80 C. CA19-9 was measured as a research assay by electrochemiluminescence immunoassay (ECLIA) using the Elecsys CA19-9 Immunoassay on a cobas e601 platform (Roche), per the manufacturer's instructions. The resulting CA19-9 values ranges from 0-793,700 U/mL (median 18.165 U/mL).

Machine Learning Data Analysis

The present machine learning-based development of a PDAC diagnostic includes a feature selection step, a training step, and a validation step using a blinded test set. To mitigate the effects of overfitting, the blinded tests sets are separate and completely independent from the data used to discover features or to train the model. First, a features' selection was performed using Least Absolute Shrinkage and Selection Operator (LASSO) on the 14-biomarker-candidates from the training set of data, which is labeled with each subject's true state (for example, those with PDAC versus those without PDAC). Using these identified features, a classifier model was then trained. During the development of this model, its performance was evaluated using cross validation within the training set. Finally, this machine learning model was evaluated by classifying subjects in a separate, user-blinded test set.

The following additional steps were taken to mitigate the effects of overfitting in the development of and the evaluation of the presently disclosed machine learning model. Instead of using only a single machine learning algorithm, which can overfit to artifacts in the data that will not be present in prospective datasets, an ensemble of classifier models (including K-Nearest-Neighbors, SVM, linear discriminate analysis, logistic regression, and Naive Bayes) was used and their results were averaged. By performing model averaging, the overfitting by any single algorithm can be mitigated, as each model will overfit to the data differently and thus be averaged out, providing a more accurate model than any single method alone (24). Additionally, a bootstrapping method was applied to randomly select multiple subgroups of the training set to train the ensemble model, and thus mitigate the effects of outlier data in the training set. Most importantly, the model was evaluated using an independent, blinded data set only once, avoiding the possibility of the model overfitting the test set. The classifier model implemented in Python and LASSO was carried out in Matlab 2017a.

### Example 1

### Biomarker Panel Development

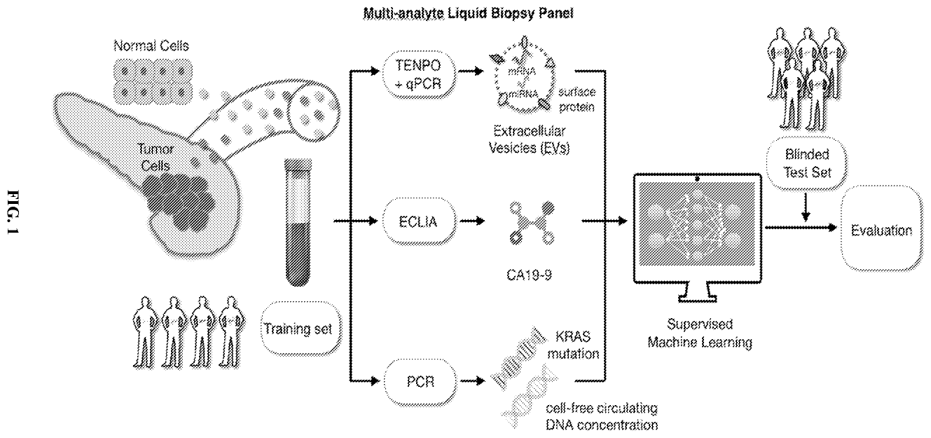

A biomarker panel was constructed a including multiple blood-based analytes with the aim of improving sensitivity and specificity of disease diagnosis and staging (FIG. 1). Previously reported tumor-associated markers were included such as ccfDNA concentration and ccfDNA-based detection of the KRAS G12D, V, and R mutations present in about 90% of PDAC tumors (25). CA19-9 is a routinely ordered laboratory test for PDAC monitoring and thus could readily be applied in the setting of disease detection. To determine which miRNAs would be optimal for analyzing human samples, EVs and their miRNA cargo were isolated from the plasma of a discovery cohort of 29 patients (FIG. 5 and FIG. 9), including 7 healthy controls, 5 disease controls (1 non-malignant biliary stricture and 4 pancreatitis), and 17 PDAC patients of various disease stages. Next-generation sequencing was performed on extracted EV miRNA and we applied the LASSO feature to the results to identify the most informative miRNAs (FIG. 6A, 6B). Among the 8 most informative, only 5 were selected to move forward based on their abundance as detected by qPCR (Cq≤40, FIG. 6C). To validate qPCR-based detection of the 5 miRNAs, matched samples were run by qPCR and the results compared to sequencing results, resulting in a correlation coefficient of R²=0.6. Then, six EV mRNA candidates (CD63, CK18, GAPDH, H3F3A, KRAS, ODC1) were used (these previously used to distinguish metastatic PDAC patients from healthy controls (17)). Altogether, including ccfDNA concentration, circulating mutant KRAS allele fraction, and CA19-9 concentration, a total of 14 biomarker candidates were analyzed for each subject.

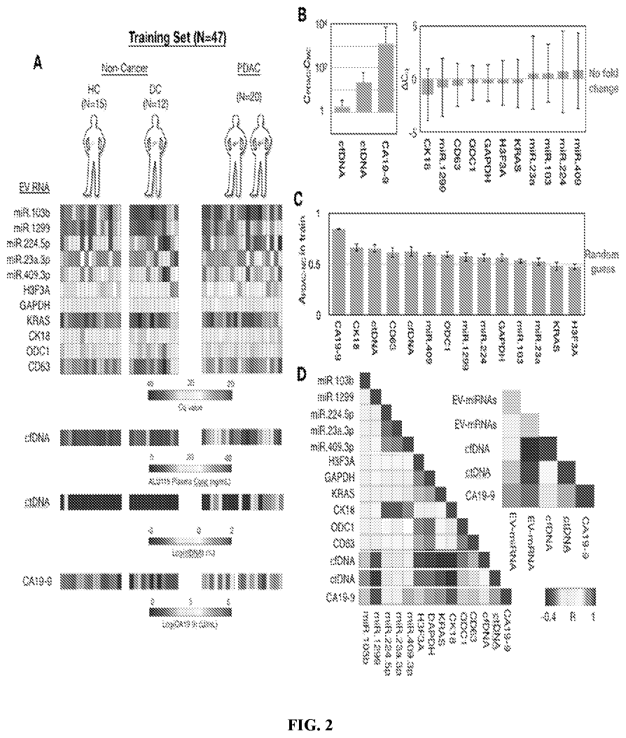

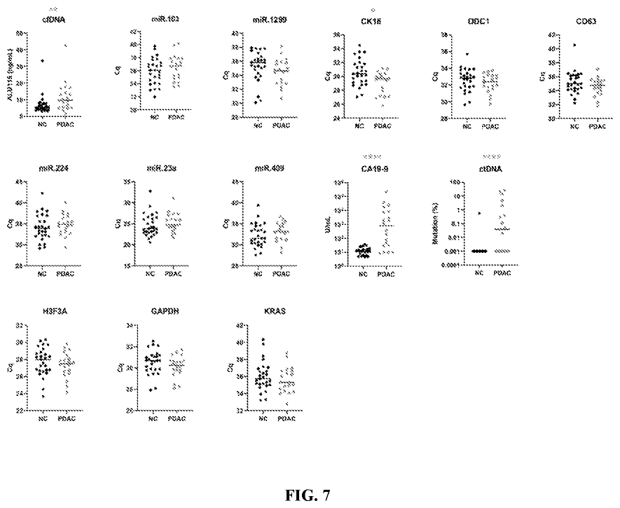

Using this panel of 14 biomarkers, a machine learning model was trained with a set of 15 healthy controls, 12 disease controls (3 IPMN and 9 pancreatitis), and 20 patients with PDAC of various stages (FIG. 2A, FIG. 5). The best individual marker at distinguishing PDAC patients from non-cancer controls was CA19-9 (FIG. 2C, FIG. 7), which also showed the highest fold change between PDAC patient cohort and non-PDAC cohort among the 14 biomarker candidates (FIG. 2B). CA19-9 achieved an accuracy of A=(TP+TN)/total=84% (95% CI 82-85%), where TP is the number of true positives and TN is the number of true negatives, using the clinical threshold of 36 U/mL(26-28). The best performing individual EV mRNA marker was CK18 (A =66%, 95% CI 58-73%), which also was shown to be a predictive marker in a previous study on EV mRNA biomarkers (17). The best performing EV miRNA marker was miR.409 (A=59%, 95% CI 55-63%), a marker that has been associated with pancreatic oncogenesis(29,30). The accuracy of ccfDNA concentration was A=62% (95% CI 52-73%), and that of circulating mutant KRAS allele fraction was A=66%.

To generate a predictive panel of biomarkers, each biomarker needs predictive power and the constituent biomarkers should not correlate with one another, such that each biomarker carries some unique information about the state of the patient. Pairwise correlation coefficients (R) between biomarkers were calculated and revealed that individual biomarkers were generally not well correlated with one another, except between CA19-9 and circulating mutant KRAS allele fraction (|R|=0.73) (FIG. 2D), and were therefore suitable to be combined together in a panel. More specifically, CA19-9 did not correlate with either ccfDNA concentration or EV RNAs (|R|<0.4). Moreover, ccfDNA concentration did not correlate with EV RNAs (|R|<0.5) and was weakly correlated with circulating mutant KRAS allele fraction |R|=0.55. Tumor derived EV miRNAs weakly correlated with one another (averaged |R| among EV miRNAs is 0.65) but not with other biomarkers (|R|<0.40). Tumor derived EV mRNAs weakly correlated with one another (averaged |R|=0.66) but not with other biomarkers (|R|<0.40). Interestingly, EV-CK18, in addition to having the greatest accuracy of any individual EV mRNA biomarker, was also particularly uncorrelated with any other measured biomarkers (|R|<0.55).

### Example 2

### Distinguishing PDAC Patients from Non-Cancer Controls

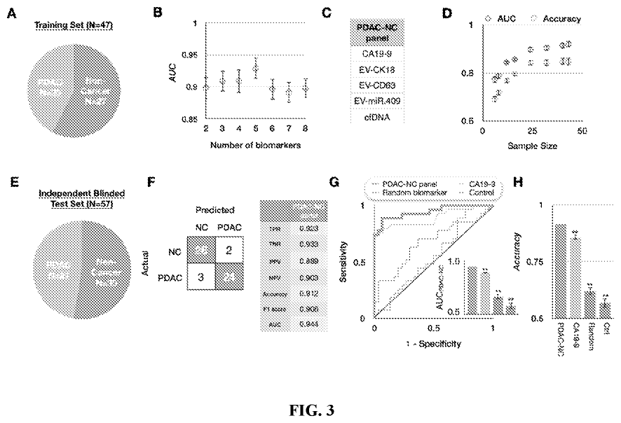

Next, to identify the optimal panel of biomarkers from the 14 discussed above to distinguish PDAC patients from non-cancer controls, LASSO was applied to the training set of data (FIG. 2A and FIG. 3A) and determined that the best performing panel (AUC=0.93), as measured using 10-fold cross-validation, included five diverse biomarkers: EV-CK18 mRNA, EV-CD63 mRNA, EV-miR.409, ccfDNA concentration, and CA19-9 (FIGS. 3A-3C). Next, the question of whether enough subjects were included to properly train the present model by generating a learning curve (FIG. 3D) was addressed. The results showed that the model's performance plateaued beyond 25 patients, indicating that the present training set sample of 47 subjects was sufficient.

To further evaluate this approach, 5-marker panel were applied to an independent blinded test set of 57 subjects (FIG. 3E) and achieved an accuracy of A=91% (FIG. 3F). Also, an AUC of 0.94 (FIG. 3G) was calculated, which was significantly better than the performance of CA19-9 alone (AUC=0.89, P<0.01). To validate that the performance is specific to the set of biomarkers that were selected, this result was compared to a control experiment where randomly chosen sets of 5 biomarkers (AUC 0.62) were evaluated. To confirm that the performance is specific to the signature of biomarkers identified by training the presently disclosed machine learning algorithm, we randomly shuffled the labels in the training set. This control experiment resulted in an AUC=0.57, equivalent to random guessing. The presently disclosed model's performance was significantly better than using randomly selected features (P<0.01) or randomly shuffled labels (P <0.01). The present model's accuracy of 91%, also outperformed CA19-9 (A=86%, P<0.01; FIG. 3H). Taken together, these results suggest that a multi-analyte panel outperforms any single biomarker for the blood-based detection of PDAC.

### Example 3

### Distinguishing Metastatic from Non-Metastatic PDAC

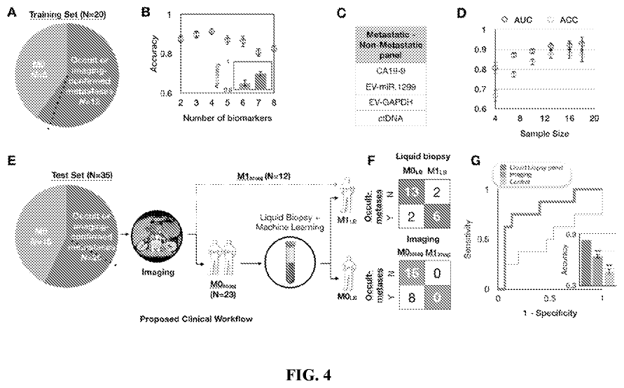

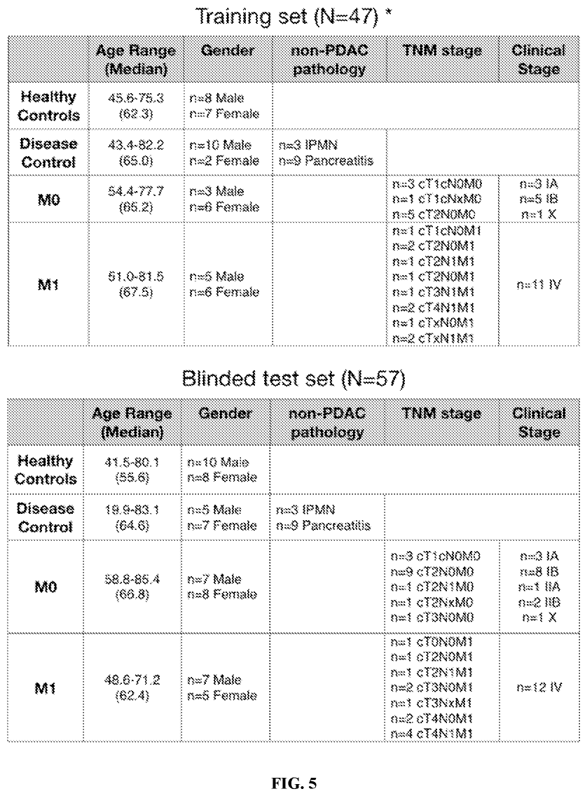

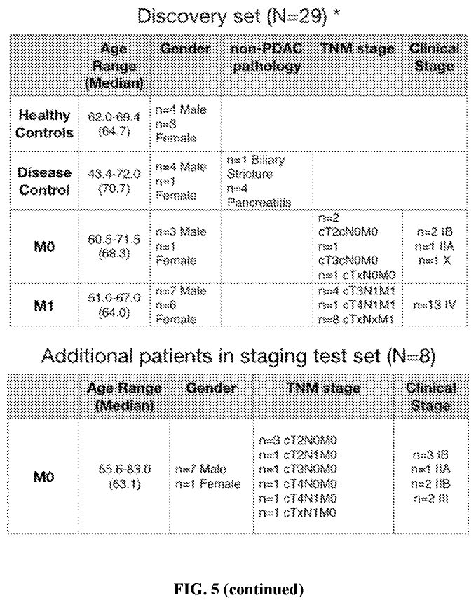

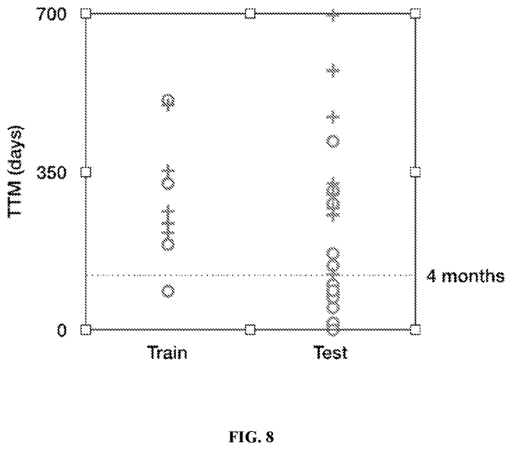

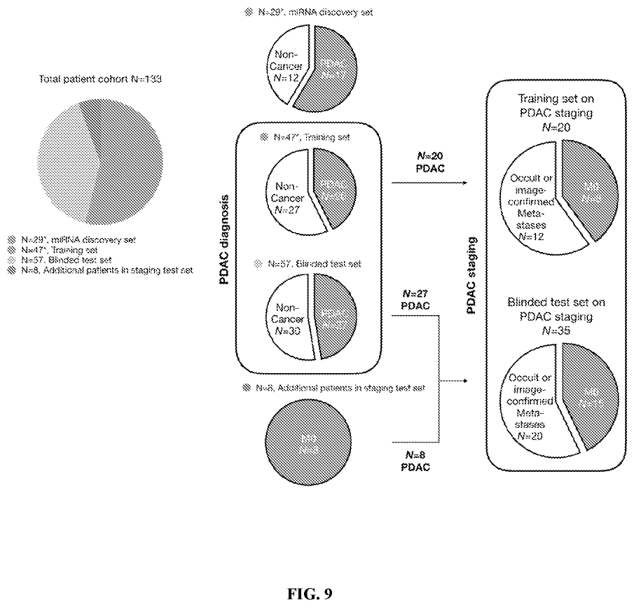

Imaging is a widely used but imperfect technique for detecting metastases and determining whether a PDAC patient's disease is sufficiently localized for consideration of curative-intent surgery. The model disclosed herein was tested to assess if it can identify a biomarker panel that, in conjunction with imaging, could better stage PDAC patients by distinguishing metastatic from non-metastatic disease. To train the model, 20 PDAC patients, originally staged by imaging, were selected which included 9 patients with no detectable metastasis (M0; including 7 resectable and 2 locally advanced), and 11 patients with metastasis (M1) (FIG. 4A). Since some patients originally identified as M0 may have had occult metastases below the level of imaging detection, a chart review was conducted and retrospectively the M0 patients were re-stratified into two groups: 1) M0s: those with no evidence of metastatic disease intraoperatively or within 4 months of follow-up and 2) Occult metastases: those who had metastases detected intraoperatively or had metastatic recurrence within 4 months of blood draw. A sensitivity analysis of time-to-distant-failure was performed among the patient cohort (FIG. 8) to select the cutoff of 4 months, a time that is far shorter than the median recurrence-free, relapse-free, or metastasis-free survivals reported in both experimental and control arms in large randomized trials (31-33). This stratification resulted in the training set of 8 M0 and 12 M1 (11 with imaging-confirmed metastases and one with occult metastases) (FIG. 4A). Using LASSO, a biomarker panel of 4 markers, including EV-miR.1299, EV-GAPDH, circulating mutant KRAS allele fraction, and CA19-9 was selected as having the highest Accuracy (A=91%; FIG. 4B, C). A learning curve using 8-fold cross validation showed that the curve plateaued by 15 subjects, indicating that the 20 subjects in the current training set were sufficient (FIG. 4D).

To further evaluate the panel's ability to identify occult metastatic disease, the approach to an independent blinded test set of 35 subjects with PDAC was applied as part of a clinical workflow starting with standard of care diagnostic imaging and followed by liquid biopsy (FIG. 4E). Twelve of 35 patients were identified by imaging alone as having metastases, were classified as Ml, and had no further evaluation. The remaining 23 patients were determined by baseline imaging to have no detectable metastases (MO-imaging). Upon retrospective chart review, 15 of 23 had no evidence of metastases within 4 months (median time to metastases. Eight of 23 patients were determined to have had occult metastases, including 4 who had surgery aborted due to intraoperative detection of metastatic disease and another 4 who completed surgery but had distant metastases detected on imaging within 4 months of their baseline blood draw. The liquid biopsy workflow correctly identified 6 of 8 patients as having metastatic disease, and 13 of 15 patients as being metastasis-free. Thus, by comparing the liquid biopsy prediction to the true state of the patients, the ptest had an accuracy of detecting distant metastasis of A =83% (19/23) with sensitivity of 75% and specificity of 87% (AUC=0. 8), which compares favorably to the accuracy of imaging alone (A=65% (15/23); P<0.01. FIG. 4F) among 23 patients originally identified as M0 by imaging.

We also ran a control experiment to confirm the performance is specific to the biomarkers identified from the disclosed training set. In the control experiment, the labels in the training set were randomly shuffled and the resulting AUC=0.49 with accuracy of 46%, was equivalent to random guessing. The presently disclosed model's performance was significantly better than the control experiment (P<0.01).

### Example 4

### Discussion

Disclosed herein are methods for assessing pancreatic cancer in a subject based upon a multi-analyte panel that algorithmically combines tumor-associated EV mRNA and miRNA, DNA (ccfDNA concentration and KRAS mutation detection), and CA19-9 using machine learning. Using training sets of samples from patients, disease controls, and healthy individuals as well as independent, blinded test sets, this approach was used first used to distinguish cancer versus non-cancer patient samples. Next, the model for disease staging and the detection of metastatic disease for PDAC patients originally staged by standard of care imaging were re-trained.

In the present study, a multi-analyte liquid biopsy approach was applied to clinical baseline blood samples obtained from patients with PDAC of all stages, as well as healthy and disease controls. The disclosed platform was shown to be able to accurately identify patients with PDAC (A=91%) and, for patients with pathologically confirmed PDAC, improve the detection of occult metastases that are not initially detected by standard of care imaging but are found intraoperatively or shortly after surgery (A=83%). Surgical resection remains the only curative therapy for PDAC (3),but is limited to patients without detectable metastases. At time of diagnosis, approximately 40% of PDAC patients will have locally advanced disease, typically treated with systemic therapy with the goal of down-staging the tumor such that the patient becomes a candidate for curative intent surgery. Only about 15 -20% of patients will be deemed candidates for surgical resection at the time of diagnosis based on imaging and clinical status (1,3). Even in this subgroup, the intraoperative detection of metastases, prompting the surgery to be aborted, or rapid emergence of distant metastases within months of surgery, can still occur (1,3,34-36). Those patients with recurrent disease demonstrate survival similar to a de novo metastatic patient (37) thus questioning the potential benefit of surgery in that setting. This yields two important clinical problems that the present approach addresses: 1) detecting disease at an early enough stage for surgery to be feasible, and 2) once diagnosed with PDAC, accurately determining which patients would or would not benefit from surgery.

This work differentiates itself most significantly from previous work in the following aspects: 1) it combines a diverse set of non-invasive markers, 2) the disclosed biomarkers panel can not only diagnose PDAC, but also improve staging accuracy; and 3) it uses machine learning approaches that are resilient against overfitting and can continue to be trained and improved in future studies. To construct the presently disclosed multi-analyte panel, the marker CA19-9, which is routinely ordered as a clinical blood test for PDAC patients, with existing liquid biopsy approaches for measuring ccfDNA concentration (10,38,39); ccfDNA allele fraction of mutant KRAS(40,41), and mRNA and miRNA isolated from tumor-associated EVs were selected. The accuracy of single-analyte CA19-9 (84%) and KRAS mutation detection (66%) in the present cohort is consistent with previous publication (83% and 67% respectively)(34). Previous investigations have shown that the mRNA and miRNA cargo of tumor derived EVs can be readily detected in pre-clinical and clinical samples(35). The present findings demonstrate that EV transcriptional profiling provides orthogonal diagnostic information, thus providing the rationale for adding EV-based measures to those from protein- and DNA-based markers.

In various investigations, multi-analyte panels have demonstrated several key advantages compared to single markers (16,18). Individual EV biomarkers have previously demonstrated promising results for PDAC (36,42-45), but have faced challenges when applied to patient cohorts in different institutions (46). For example, Melo et al. reported that GPC1⁺ exosomes were informative for distinguishing early and late stage PDAC patients from healthy and disease controls with an AUC=1(36). However, independent studies reported markedly different performance of GPC1⁺ EVs for PDAC diagnosis(42,46). Several recent publications have also shown a benefit of combining multiple biomarkers for PDAC diagnosis, however, biomarkers in most publications tend to come from a single category, e.g., from EV cargo nucleic acids including miRNAs (47-49), mRNAs(17), DNAs(50), or from EV surface protein profiling (15). Few studies combined biomarkers from different categories: Cohen et al combined CA19-9 with circulating tumor DNA and plasma proteins (19); Madhavan et al combined EV cargo proteins and miRNAs(21), but both focused on PDAC diagnosis only. The presently disclosed assays, which have identified signatures across multiple biomarkers, have the potential to be more robust for diverse patient populations and are less dependent on any single reagent than assays built around a single marker. Nevertheless, one potential drawback to a multi-analyte panel could be a requirement for a large total blood volume. However, the presently disclosed entire panel only requires 3 mL plasma, less than the typical yield from a standard 10 mL blood collection tube.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### REFERENCES

1. Society A C. Key statistics for pancreatic cancer [Internet]. 2019. Available from: https://www.cancer.org/cancer/pancreatic-cancer/about/key-statistics.html
2. Hidalgo M. Pancreatic cancer. N Engl J Med [Internet]. Mass Medical Soc; 2010; 362:1605-17. Available from: https://www.nejm.org/doi/full/10.1056/NEJMra0901557
3. Ryan D P, Hong T S, Bardeesy N. Pancreatic adenocarcinoma. N Engl J Med [Internet]. Mass Medical Soc; 2014; 371:1039-49. Available from: https://www.nejm. org/doi/ful1/10.1056/NEJMra1404198
4. Wolff R A, Varadhachary G R, Evans D B. Adjuvant therapy for adenocarcinoma of the pancreas: analysis of reported trials and recommendations for future progress. Ann Surg Oncol [Internet]. Springer; 2008; 15:2773. Available from: http://gateway.webofknowledge.com/gateway/Gateway. cgi?GWVersion=2&SrcApp=GSSearch&SrcAuth=Scholar&DestApp=WOS_CPL&DestLinkType=CitingArticles &UT=000259186300022&SrcURL=https://scholar.google. com/&SrcDesc=Back+to+Google+Scholar&GSPage=TC
5. Wolff R A. Adjuvant or neoadjuvant therapy in the treatment in pancreatic malignancies: where are we. Surg Clin [Internet]. Elsevier; 2018; 98:95-111. Available from: http://gateway.webofknowledge.com/gateway/Gateway.cgi?GWVersion=2&SrcApp=GS S earch&SrcAuth=Scholar&DestApp=WOS_CPL&DestLinkType=CitingArticles &UT=000419261300011&SrcURL=https://scholar.google.com/&SrcDesc=Back+to+Google+Scholar&GSPage=TC
6. Crowley E, Di Nicolantonio F, Loupakis F, Bardelli A. Liquid biopsy: monitoring cancer-genetics in the blood. Nat Rev Clin Oncol [Internet]. Nature Publishing Group; 2013; 10:472. Available from: https://www.gene-quantification.de/crowley-et-al-cancer-genetics-liquid-biopsy-review-2013.pdf
7. Bettegowda C, Sausen M, Leary RJ, Kinde I, Wang Y, Agrawal N, et al. Detection of circulating tumor DNA in early- and late-stage human malignancies. Sci Transl Med. 2014;
8. Li J, Zhu J, Hassan M M, Evans D B, Abbruzzese J L, Li D. K-ras mutation and p16 and preproenkephalin promoter hypermethylation in plasma DNA of pancreatic cancer patients: in relation to cigarette smoking. Pancreas [Internet]. NIH Public Access; 2007; 34:55. Available from: https://www.ncbi.nlm.nih.gov/pmearticles/PMC1905887
9. Bergquist J R, Puig C A, Shubert C R, Groeschl R T, Habermann E B, Kendrick M L, et al. Carbohydrate antigen 19-9 elevation in anatomically resectable, early stage pancreatic cancer is independently associated with decreased overall survival and an indication for neoadjuvant therapy: A national cancer database study. J Am Coll Surg. 2016;
10. Benesova L, Belsanova B, Suchanek S, Kopeckova M, Minarikova P, Lipska L, et al. Mutation-based detection and monitoring of cell-free tumor DNA in peripheral blood of cancer patients. Anal. Biochem. 2013.
11. Da Silva Filho B F, Gurgel A P A D, De Freitas Lins Neto M A, De Azevedo D A, De Freitas A C, Da Costa Silva Neto J, et al. Circulating cell-free DNA in serum as a biomarker of colorectal cancer. J Clin Pathol. 2013;
12. Tsiatis A C, Norris-Kirby A, Rich R G, Hafez M J, Gocke C D, Eshleman J R, et al. Comparison of Sanger sequencing, pyrosequencing, and melting curve analysis for the detection of KRAS mutations: Diagnostic and clinical implications. J Mol Diagnostics. 2010;
13. Thierry A R, Mouliere F, El Messaoudi S, Mollevi C, Lopez-Crapez E, Rolet F, et al. Clinical validation of the detection of KRAS and BRAF mutations from circulating tumor DNA. Nat Med. 2014;
14. Kim J, Bamlet W R, Oberg A L, Chaffee K G, Donahue G, Cao X J, et al. Detection of early pancreatic ductal adenocarcinoma with thrombospondin-2 & CA19-9 blood markers. Sci Transl Med. 2017;
15. Yang K S, Im H, Hong S, Pergolini I, Del Castillo A F, Wang R, et al. Multiparametric plasma EV profiling facilitates diagnosis of pancreatic malignancy. Sci Transl Med. 2017;
16. Kinde I, Wu J, Papadopoulos N, Kinzler K W, Vogelstein B. Detection and quantification of rare mutations with massively parallel sequencing. Proc Natl Acad Sci USA. 2011;
17. Ko J, Bhagwat N, Yee SS, Ortiz N, Sahmoud A, Black T, et al. Combining machine learning and nanofluidic technology to diagnose pancreatic cancer using exosomes. ACS Nano [Internet]. ACS Publications; 2017; 11:11182-93. Available from: https://pubs.acs.org/doi/full/10.1021/acsnano.7b05503
18. Cohen J D, Li L, Wang Y, Thoburn C, Afsari B, Danilova L, et al. Detection and localization of surgically resectable cancers with a multi-analyte blood test. Science (80-). 2018;
19. Cohen J D, Javed A A, Thoburn C, Wong F, Tie J, Gibbs P, et al. Combined circulating tumor DNA and protein biomarker-based liquid biopsy for the earlier detection of pancreatic cancers. Proc Natl Acad Sci. 2017;
20. McShane L M, Altman D G, Sauerbrei W, Taube S E, Gion M, Clark G M. REporting recommendations for tumor MARKer prognostic studies (REMARK). Breast Cancer Res Treat. 2006;
21. Madhavan B, Yue S, Galli U, Rana S, Gross W, Muller M, et al. Combined evaluation of a panel of protein and miRNA serum-exosome biomarkers for pancreatic cancer diagnosis increases sensitivity and specificity. Int J cancer [Internet]. Wiley Online Library; 2015; 136:2616-27. Available from: https://onlinelibrary.wiley.com/doi/full/10.1002/ij c.29324
22. Love M I, Huber W, Anders S. Moderated estimation of fold change and dispersion for RNA-seq data with DESeq2. Genome Biol [Internet]. BioMed Central; 2014; 15:550. Available from: https://genomebiology.biomedcentral.com/articles/10.1186/s13059-014-0550-8
23. Zhu Q, Fisher SA, Shallcross J, Kim J. VERSE: a versatile and efficient RNA-Seq read counting tool. bioRxiv [Internet]. Cold Spring Harbor Laboratory; 2016; 53306. Available from: https://www.biorxiv.org/content/biorxiv/early/2016/05/14/053306.full. pdf
24. Statnikov A, Aliferis C F, Tsamardinos I, Hardin D, Levy S. A comprehensive evaluation of multicategory classification methods for microarray gene expression cancer diagnosis. Bioinformatics [Internet]. Oxford University Press; 2004; 21:631-43. Available from: https://academic.oup.com/bioinformatics/article/21/5/631/219898
25. Waters A M, Der C J. KRAS: The critical driver and therapeutic target for pancreatic cancer. Cold Spring Harb Perspect Med. 2018;
26. Rifts Jr R E, del Villano B C, Go V L W, Herberman R B, Klug T L, Zurawski Jr V R. Initial clinical evaluation of an immunoradiometric assay for CA 19-9 using the NCI serum bank. Int J cancer [Internet]. Wiley Online Library; 1984; 33:339-45. Available from: https://onlinelibrary.wiley.com/doi/pdf/10.1002/ij c.2910330310
27. Farini R, Fabris C, Bonvicini P, Piccoli A, Del Favero G, Venturini R, et al. C A 19-9 in the differential diagnosis between pancreatic cancer and chronic pancreatitis. Eur J Cancer Clin Oncol [Internet]. Elsevier; 1985;21:429-32. Available from: http://gateway.webofknowledge.com/gateway/Gateway.cgi?GWVersion=2&SrcApp=GSSearch&SrcAuth=Scholar&DestApp=WOS_CPL&DestLinkType=CitingArticles &UT=A1985AGX2400005&SrcURL=https://scholar.google.com/&SrcDesc=Back+to +Google+Scholar&GSPage=TC
28. Safi F, Roscher R, Beger H G. The clinical relevance of the tumor marker CA 19-9 in the diagnosing and monitoring of pancreatic carcinoma. Bull Cancer [Internet]. 1990; 77:83-91. Available from: http://gateway.webofknowledge.com/gateway/Gateway.cgi?GWVersion=2&SrcApp=GSSearch&SrcAuth=Scholar&DestApp=WOS_CPL&DestLinkType=CitingArticles &UT=A1990CM13100010&SrcURL=https://scholar.google.com/&SrcDesc=Back+to+Google+Scholar&GSPage=TC
29. Drakaki A, Iliopoulos D. MicroRNA-gene signaling pathways in pancreatic cancer. Biomed J [Internet]. Elsevier Limited; 2013; 36. Available from: http://biomedj.cgu.edu.tw/pdfs/2013/36/5/images/BiomedJ_2013_36_5_200_119690.pdf
30. Bloomston M, Frankel W L, Petrocca F, Volinia S, Alder H, Hagan J P, et al. MicroRNA expression patterns to differentiate pancreatic adenocarcinoma from normal pancreas and chronic pancreatitis. Jama [Internet]. American Medical Association; 2007; 297:1901-8. Available from: https://jamanetwork.com/journals/jama/articlepdf/206899/jpc70005_1901_1908.pdf
31. Neoptolemos J P, Stocken D D, Friess H, Bassi C, Dunn J A, Hickey H, et al. A Randomized Trial of Chemoradiotherapy and Chemotherapy after Resection of Pancreatic Cancer. N Engl J Med. 2004;
32. Neoptolemos J P, Moore M J, Cox T F, Valle J W, Palmer D H, McDonald A C, et al. Effect of adjuvant chemotherapy with fluorouracil plus folinic acid or gemcitabine vs observation on survival in patients with resected periampullary adenocarcinoma: The ESPAC-3 periampullary cancer randomized trial. JAMA—J Am Med Assoc. 2012;
33. Conroy T, Hammel P, Hebbar M, Ben Abdelghani M, Wei A C, Raoul J-L, et al. FOLFIRINOX or Gemcitabine as Adjuvant Therapy for Pancreatic Cancer. N Engl J Med. 2018;
34. Sefrioui D, Blanchard F, Toure E, Basile P, Beaussire L, Dolfus C, et al. Diagnostic value of CA19. 9, circulating tumour DNA and circulating tumour cells in patients with solid pancreatic tumours. Br J Cancer [Internet]. Nature Publishing Group; 2017; 117:1017. Available from: https://www.nature.com/articles/bjc2017250
35. Ko J, Carpenter E, Issadore D. Detection and isolation of circulating exosomes and microvesicles for cancer monitoring and diagnostics using micro-/nano-based devices. Analyst [Internet]. Royal Society of Chemistry; 2016;141:450-60. Available from: https://pubs.rsc.org/en/content/articlehtml/2015/an/c5an01610j
36. Melo S A, Luecke L B, Kahlert C, Fernandez A F, Gammon S T, Kaye J, et al. Glypican-1 identifies cancer exosomes and detects early pancreatic cancer. Nature. 2015;
37. Gbolahan O B, Tong Y, Sehdev A, O'neil B, Shanda S. Overall survival of patients with recurrent pancreatic cancer treated with systemic therapy: A retrospective study. BMC Cancer. BMC Cancer; 2019; 19:1-9.
38. Fawzy A, Sweify K M, El-Fayoumy H M, Nofal N. Quantitative analysis of plasma cell-free DNA and its DNA integrity in patients with metastatic prostate cancer using ALU sequence. J Egypt Natl Canc Inst. 2016;
39. Umetani N, Kim J, Hiramatsu S, Reber H A, Hines O J, Bilchik A J, et al. Increased integrity of free circulating DNA in sera of patients with colorectal or periampullary cancer: Direct quantitative PCR for ALU repeats. Clin Chem. 2006;
40. Kamisawa T, Wood L D, Itoi T, Takaori K. Seminar Pancreatic cancer. Lancet. 2016;
41. Allenson K, Castillo J, San Lucas F A, Scelo G, Kim D U, Bernard V, et al. High prevalence of mutant KRAS in circulating exosome-derived DNA from early-stage pancreatic cancer patients. Ann Oncol. 2017;
42. Li T Da, Zhang R, Chen H, Huang Z P, Ye X, Wang H, et al. An ultrasensitive polydopamine bi-functionalized SERS immunoassay for exosome-based diagnosis and classification of pancreatic cancer. Chem Sci. 2018;
43. Liang K, Liu F, Fan J, Sun D, Liu C, Lyon C J, et al. Nanoplasmonic quantification of tumour-derived extracellular vesicles in plasma microsamples for diagnosis and treatment monitoring. Nat Biomed Eng [Internet]. Nature Publishing Group; 2017; 1:21. Available from: https://www.ncbi.nlm.nih.gov/pmc/articles/PMC5543996
44. Joshi G K, Deitz-McElyea S, Liyanage T, Lawrence K, Mali S, Sardar R, et al. Label-free nanoplasmonic-based short noncoding RNA sensing at attomolar concentrations allows for quantitative and highly specific assay of microRNA-10b in biological fluids and circulating exosomes. ACS Nano [Internet]. ACS Publications; 2015; 9:11075-89. Available from: https://pubs.acs.org/doi/full/10.1021/acsnano.5b04527
45. Takahasi K, Iinuma H, Wada K, Minezaki S, Kawamura S, Kainuma M, et al. Usefulness of exosome-encapsulated microRNA-451a as a minimally invasive biomarker for prediction of recurrence and prognosis in pancreatic ductal adenocarcinoma. J Hepato-Biliary-Pancreatic Sci [Internet]. Wiley Online Library; 2018; 25:155-61. Available from: https://onlinelibrary.wiley.com/doi/pdf/10.1002/jhbp.524
46. Lucien F, Lac V, Billadeau D D, Borgida A, Gallinger S, Leong H S. Glypican-1 and glycoprotein 2 bearing extracellular vesicles do not discern pancreatic cancer from benign pancreatic diseases. Oncotarget [Internet]. Impact Journals, LLC; 2019; 10:1045. Available from: https://www.ncbi.nlm.nih.gov/pmearticles/PMC6383691
47. Lai X, Wang M, McElyea S D, Sherman S, House M, Korc M. A microRNA signature in circulating exosomes is superior to exosomal glypican-1 levels for diagnosing pancreatic cancer. Cancer Lett [Internet]. Elsevier; 2017; 393:86-93. Available from: https://www.sciencedirect.com/science/article/pii/S0304383517301283
48. Goto T, Fujiya M, Konishi H, Sasajima J, Fujibayashi S, Hayashi A, et al. An elevated expression of serum exosomal microRNA-191, - 21, -451a of pancreatic neoplasm is considered to be efficient diagnostic marker. BMC Cancer. 2018;
49. Machida T, Tomofuji T, Maruyama T, Yoneda T, Ekuni D, Azuma T, et al. miR-1246 and miR-4644 in salivary exosome as potential biomarkers for pancreatobiliary tract cancer. Oncol Rep [Internet]. Spandidos Publications; 2016; 36:2375-81. Available from: https://www.spandidos-publications.com/or/36/4/2375
50. Yang S, Che SPY, Kurywchak P, Tavormina J L, Gansmo L B, Correa de Sampaio P, et al. Detection of mutant KRAS and TP53 DNA in circulating exosomes from healthy individuals and patients with pancreatic cancer. Cancer Biol Ther [Internet]. Taylor & Francis; 2017; 18:158-65. Available from: https://www.tandfonline.com/doi/full/10.1080/15384047.2017.1281499

What is claimed:

1. A method of determining whether a subject suffers from a disease or a condition, the method comprising:
a. measuring, in a processed sample from the subject, a set of circulating biomarkers comprising an extra-cellular vesicle (EV) miRNA, an EV mRNA, a circulating cell-free DNA, a circulating tumor DNA, and a protein biomarker specific for the disease or the condition;
b. applying a machine learning algorithm on the set of circulating biomarkers to generate an output indicative of a disease or a condition state of the subject;
c. determining whether the subject has the disease or the condition based upon the output so generated; and
d. treating the subject as needed.

2. A method of classifying a stage of a disease or a condition in a subject in need thereof, the method comprising:
a. measuring, in a processed sample from the subject, a set of circulating biomarkers comprising an extra-cellular vesicle (EV) miRNA, an EV mRNA, a circulating cell-free DNA, a circulating tumor DNA, and a protein biomarker specific for the disease or the condition;
b. applying a machine learning algorithm on the set of circulating biomarkers to generate an output indicative of the stage of the disease or the condition of the subject;
c. determining the stage of the disease or the condition in the subject based upon the output so generated; and
d. recommending treatment or surgery for the subject.

3. A method of assessing the efficacy of a therapy for treating a disease or a condition in a subject, the method comprising:
a. measuring, in a first processed sample taken from the subject before treatment, a set of circulating biomarkers comprising an extra-cellular vesicle (EV) miRNA, an EV mRNA, a circulating cell-free DNA, a circulating tumor DNA, and a protein biomarker specific for the disease or the condition;
b. measuring, in a second processed sample taken from the subject during or after treatment, the same set of circulating biomarkers from step (a);
c. applying a machine learning algorithm on the circulating biomarkers from step (a) and step (b) to generate a first output and a second output respectively indicative of a stage of the disease or the condition in the subject; and
d. determining a differential between the first output and second output, thereby assessing whether the efficacy of the therapy for treating the disease or the condition in the subject.

4. A method of determining whether a subject suffers from a disease or a condition, the method comprising:
a. measuring, in a processed sample from the subject, a set of a plurality of circulating biomarkers selected by machine learning such that each biomarker is indicative of the disease or condition and such that the correlation between the circulating biomarkers is minimized;
b. generating an output, optionally by a machine learning algorithm, that is indicative of a disease or a condition state of the subject;
c. determining whether the subject has the disease or the condition based upon the output so generated; and
d. treating the subject as needed.

5. The method of claim 4, wherein the correlation between the circulating biomarkers is less than 0.6.

6. The method of claim 1, wherein the determining whether a subject suffers from a disease or a condition has an accuracy of at least 90%.

7. The method of any one of the preceding claims, wherein the disease or the condition is a cancer.

8. The method of claim 7, wherein the cancer is a pancreatic cancer.

9. The method of any of one of claims 7-8, wherein the cancer is at a metastatic stage.

10. The method of any of one of claims 7-9, wherein an absence of metastasis is an indication that a treatment or a surgery is beneficial for the subject.

11. The method of any one of the preceding claims, wherein the EV miRNA comprises hsa.miR.103b, hsa.miR.23a.3p, hsa.miR.409.3p, hsa.miR.224.5p and hsa.miR.1299.

12. The method of any one of the preceding claims, wherein the EV mRNA comprises CD63, CK18, GAPDH, H3F3A, KRAS and ODC1.

13. The method of any one of the preceding claims, wherein the ccfDNA comprises an ALU repetitive element.

14. The method of any one of the preceding claims, wherein the ctDNA comprises a mutated KRAS DNA with mutation KRASG12D, KRASG12V or KRASG12R.

15. The method of any one of the preceding claims, wherein the protein biomarker is a cancer antigen protein.

16. The method of any one of the preceding claims, wherein the protein biomarker is cancer antigen 19-9 (CA19-9) protein.

17. The method of any one of the preceding claims, wherein the circulating biomarkers comprise at least hsa.miR.1299, GAPDH mRNA, a mutated KRAS DNA and CA19-9 protein.

18. The method of any one of the preceding claims, wherein the processed sample is taken from whole blood or plasma.

19. The method of any one of the preceding claims, wherein the processed sample comprises extracted, amplified and/or labeled DNA, RNA or protein.

20. The method of any one of the preceding claims, wherein the EV miRNA and EV mRNA are extracted by a track etched magnetic nanopore (TENPO) device.

21. The method of any one of the preceding claims, wherein one or more of the circulating biomarkers are measured by one or more of sequencing, quantitative PCR, digital PCR, or immunoassay.

22. The method of any one of the preceding claims, wherein the machine learning algorithm distinguishes the circulating biomarkers from a control.

23. The method of claim 22, wherein the control comprises a reference value or circulating biomarkers from a healthy subject.

24. A method of determining whether a subject suffers from a disease or condition, comprising:
a. isolating a biological sample from the subject, using a magnetic separation filter device, wherein the magnetic separation filter device comprises a layer of magnetically soft material and a plurality of pores extending through the layer of magnetically soft material;
b. analyzing two or more biomarkers from the biological sample to generate an output; and
c. determining whether the subject has the disease or condition based upon the output so generated.

25. A method of diagnosing a disease or condition in a subject, comprising:
a. isolating a biological sample from the subject, using a magnetic separation filter device, wherein the magnetic separation filter device comprises a layer of magnetically soft material and a plurality of pores extending through the layer of magnetically soft material;
b. analyzing two or more biomarkers from the biological sample to generate an output; and
c. diagnosing the disease or condition in the subject based upon the output so generated.

26. A method of treating a disease or condition in a subject, comprising:
a. isolating a biological sample from the subject, using a magnetic separation filter device, wherein the magnetic separation filter device comprises a layer of magnetically soft material and a plurality of pores extending through the layer of magnetically soft material;
b. analyzing two or more biomarkers from the biological sample to generate an output;
c. diagnosing the disease or condition in the subject based upon the output so generated; and
d. administering a therapeutically effective amount of a drug suitable for treating the disease or condition to the subject.

27. The method of any one of claims 24-26, wherein the magnetic separation filter device is a track etched magnetic nanopore (TENPO) device.

28. The method of any one of claims 24-27, wherein the pores have an average diameter ranging from about 100 nm to 100 μm.

29. The method of claim 28, wherein the pores have an average diameter ranging from about 500 nm to about 25 μm.

30. The method of any one of claims 24-29, wherein the magnetic separation filter device comprises at least 1000 pores/mm².

31. The method of any one of claims 24-30, wherein the magnetically soft material comprises a nickel-iron alloy.

32. The method of any one of claims 24-31, wherein the magnetic separation filter device further comprises a layer comprising a material chosen from nickel and gold.

33. The method of any one of claims 24-32, wherein the disease or condition is cancer.

34. The method of claim 33, wherein the cancer is a pancreatic cancer.

35. The method of claim 34, wherein the pancreatic cancer is pancreatic ductal adenocarcinoma (PDAC).

36. The method of any one of claims 33-35, wherein the cancer is metastatic.

37. The method of any one of claims 33-35, wherein the cancer is non-metastatic.

38. The method of any one of claims 33-37, wherein the biological sample comprises a plurality of extra-cellular vesicles (EV).

39. The method of claim 38, wherein the plurality of extra-cellular vesicles are specific for the disease or condition.

40. The method of any one of claims 38-39, wherein the two or more biomarkers comprises EV miRNA or EV mRNA molecules.

41. The method of claim 40, wherein the EV miRNA molecules are selected from the group consisting of hsa.miR.103b, hsa.miR.23a.3p, hsa.miR.409.3p, hsa.miR.224.5p, hsa.miR.1299, and any combinations thereof.

42. The method of claim 40, wherein the EV mRNA molecules are selected from the group consisting of CD63, CK18, GAPDH, H3F3A, KRAS, ODC1, and any combinations thereof.

43. The method of any one of claims 40-42, wherein the analyzing of the two or more biomarkers comprises measuring an amount of the EV miRNA or EV mRNA molecules.

44. The method of any one of claims 24-43, wherein the two or more biomarkers further comprises a protein biomarker.

45. The method of claim 44, wherein the protein biomarker is CA19-9 protein.

46. The method of claim 45, wherein the analyzing of the two or more biomarkers comprises measuring a concentration of the CA19-9 protein.

47. The method of any one of claims 24-46, wherein the two or more biomarkers further comprises a circulating cell-free DNA.

48. The method of claim 47, wherein the analyzing of the two or more biomarkers comprises measuring a concentration of the circulating cell-free DNA.

49. The method of any one of claims 24-48, wherein the two or more biomarkers further comprises a circulating tumor DNA.

50. The method of claim 24-49, wherein the circulating tumor DNA comprises a mutated KRAS DNA.

51. The method of claim 50, wherein the mutated KRAS DNA comprises a G12D, G12V or G12R mutation.

52. The method of any one of claims 24-51, wherein the analyzing of the two or more biomarkers comprises sequencing, quantitative PCR, digital PCR, or immunoassay.

53. The method of any one of claims 24-52, wherein the two or more biomarkers comprises an EV miRNA molecule selected from hsa.miR.103b, hsa.miR.23a.3p, hsa.miR.409.3p, hsa.miR.224.5p, and hsa.miR.1299; an EV mRNA molecule selected from CD63, CK18, GAPDH, H3F3A, KRAS, and ODC1; CA19-9 protein, a circulating cell-free DNA, a mutated KRAS DNA, or any combination thereof

54. The method of any one of claims 24-53, wherein the biological sample is taken from whole blood or plasma of the subject.

55. The method of any one of claims 24-54, further comprising applying a machine learning algorithm to the analyzing two or more biomarkers from the biological sample.

56. The method of claim 55, wherein the machine learning algorithm comprises Least Absolute Shrinkage Selection Operator (LASSO).

57. The method of claim 55, wherein the machine learning algorithm uses one or more classifier models selected from the group consisting of K-Nearest-Neighbors, SVM, linear discriminate analysis, logistic regression, Naive Bayes, and any combination thereof

58. The method of any one of claims 55-57, wherein the machine learning algorithm distinguishes at least one of the two or more biomarkers from a control.

59. The method of claim 58, wherein the control comprises a reference value or circulating biomarkers from a healthy subject.

60. The method of any one of claims 24-59, wherein the isolating of the biological sample comprise contacting the biological sample with an antibody.

61. The method of claim 60, wherein the antibody comprise anti-human CD326, anti-human CD104, anti-human c-Met Monoclonal, anti-human CD44v6 antibody, anti-human TSPAN8, or any combination thereof

62. The method of any one of claims 24-61, wherein the method has an accuracy of more than 90% in identifying the disease or condition.

63. The method of any one of claims 24-62, wherein the method has an accuracy of more than 80% in identifying metastatic status of the disease or condition.

64. The method of any one of claims 62-63, wherein the accuracy is higher than a comparable method without the isolating the biological sample using the magnetic separation filter device.
